(19) United States
(12) Patent Application Publication
(10) Pub. No.: US 2022/0119496 A1
(43) Pub. Date: Apr. 21, 2022

(54) INHIBITORS OF CYSTEINE PEPTIDASES ISOLATED FROM NATURAL RAW MATERIALS AND USE OF THE INHIBITORS IN MEDICINE AND VETERINARY MEDICINE

(71) Applicants: Andrzej Marek RAPAK, Brzeg (PL); Jacek Andrzej RAZNIEWSKI, Wroclaw (PL); Lukasz Pawel NIERADKO, Brzezina (PL); Maciej Zygmunt SIEWINSKI, Wroclaw (PL)

(72) Inventor: Maciej Zygmunt SIEWINSKI, Wroclaw (PL)

(73) Assignees: Andrzej Marek RAPAK, Brzeg (PL); Jacek Andrzej RAZNIEWSKI, Wroclaw (PL); Lukasz Pawel NIERADKO, Brzezina (PL); Maciej Zygmunt SIEWINSKI, Wroclaw (PL)

(21) Appl. No.: 17/431,160

(22) PCT Filed: Feb. 15, 2020
(86) PCT No.: PCT/PL2020/000018

§ 371 Date: Aug. 14, 2021

(30) Foreign Application Priority Data
Feb. 15, 2019 (PL) P.428931
Oct. 18, 2019 (PL) P.431519

(51) Int. Cl. C07K 14/81; A61P 35/00

(52) U.S. Cl. CPC C07K 14/8139; A61K 38/00; A61P 35/00

(57) ABSTRACT

The invention discloses low molecular weight cysteine peptidase inhibitors of natural origin, which are several-amino acid oligopeptides of the molecular weight below 3 kDa, free of salts with a molecular weight below 700 Da, a method for isolation of the same from natural organic material selected from the group consisting of egg whites, casein or milk and homogenates of plants such as knotweed, especially of the species Fallopia japonica, Houtt, mistletoe, soy, pineapple, rice, potatoes and other substances of natural origin, as well as medical (first) use in human and veterinary medicine of the low-molecular cysteine peptidase inhibitors of natural origin, being several-amino acid oligopeptides of the molecular weight below 3 kDa and the knotweed cystatin, as well as their use in manufacturing medicaments useful in prevention and treatment of inflammations associated with overexpression of cysteine peptidase enzymes originating from infecting microorganisms, or with overexpression of cysteine peptidase enzymes, being autogenic cysteine cathepsins in the human body.

The invention relates to inhibitors of cysteine peptidases isolated from natural raw materials and use of the inhibitors in medicine and veterinary medicine.

Cysteine proteinases (also known as thiol or sulfhydrole proteinases)—responsible for catalyzing the hydrolysis reaction of peptide bonds in proteins, are involved in numerous physiological and pathological processes.

Overexpression of cysteine peptidase enzymes is associated with the emergence and development of many diseases. Those processes have been studied in most details with respect to cancer diseases. In the tumor formation process, those enzymes participate at many levels: invasion, tumor transformation, angiogenesis, apoptosis and metastasis.

During degenerative diseases, in the living body the level of cysteine peptidases increases rapidly and the excess of the same cannot be inhibited due to a deficiency of autogenic inhibitors. A living organism is not able to properly replenish the deficiency of inhibitors to the level necessary to control in vivo the activity of the pathogenic enzymes.

Inhibition of activity and inhibition of overexpression of cysteine peptidases is currently a key issue in the therapy of numerous medical conditions, due to the presence of those pathogenic enzymes—both exogenous and autogenic, among others in inflammation, for example in inflammation of the mouth as well as the upper and lower respiratory tract, and also because of their described in the literature role of initiators of a number of pathogenic changes in both human and veterinary patients.

Measurement of the activity of pathogenic cysteine peptidases and their inhibitors in patients may also play an important diagnostic role, allowing to monitor the processes of dynamic changes in the level of enzymes that occur both in the disease process and in the recovery phase.

The human body is for pathogens a source of components of matter and energy the pathogens need for surviving and it results in destruction of normal cells, which in turn causes development of disease symptoms

To achieve this, pathogens must first turn off or weaken the body's defense mechanisms. Colonization of the human (or animal) body, as well as the destruction of the immune system by pathogens occurs, inter alia, by a harmful to the organism destructive action of pathogenic enzymes, including cysteine peptidases. It also turns out that the imbalance between the effect of overexpression of cysteine cathepsins and the level of their autogenous inhibitors—cystatins, weakens the function of immune cells and thus can facilitate the aggression by invasion of cancer cells or destruction of immune cells by pathogenic microorganisms. Since cysteine cathepsins appear at the inflammation sites, including tumor microenvironments, they constitute important targets in the development of new therapies. It is also suggested in the literature that the use of specific cysteine cathepsin inhibitors in cancer models in vivo may lead to beneficial changes in the host's immune system. This should be considered as increasing the therapeutic effect of the system. Furthermore, the participation of cysteine cathepsins in emerging anti-drug resistance in various therapies, including oncological ones, has been confirmed. When interference with the immune system occurs, a balance between the activity of cysteine peptidases (autogenic cathepsins or exogenous enzymes from microorganisms) and their inhibitors (cystatins) is unlikely (Jakiš T., Pišlar A., Jewett A., Kos J. (2019): Cysteine cathepsins in tumor-associated immune cells. Front Immunol. 10; 2037. doi: 10.3389/fimmu.2019.02037 and Magister S., Kos J. (2013): Cystatins in immune system. J Cancer. 4; 45-56. doi: 10.7150/jca.5044).

Extracellular cysteine cathepsins are considered to be key initiators of many important pathogenic changes in diseases such as cancer or inflammation, for example infections. Cysteine cathepsins initiate the degradation of extracellular space by specific and selective modification of extracellular proteins. Under disease conditions, the level of cysteine peptidase activity is most often deregulated, which leads to their overexpression in the extracellular space. Therefore, these enzymes are considered to be highly precise therapeutic targets for diseases associated with their over-expression. It is suggested that research and control associated with proteolysis induced by these enzymes in the extracellular space opens up many possibilities that may prove to be new therapeutic directions. Understanding the action and ability to control the activity of cysteine cathepsins and enzymes secreted by pathogenic microorganisms also opens up new opportunities in translational medicine associated with the observation that in parallel with the increase in life expectancy, the incidence of many diseases also increases.

It is also known that cysteine cathepsins support the transformation and migration of cancer cells, and also increase the invasiveness (aggressiveness) of particular types of cancer. This means that abnormalities in their activity and expression may be associated with features of tumor aggression. It turns out that these enzymes in the tumor micro-environment are involved in the proliferation, invasion and metastasis of cancer cells. They also participate in the degradation of extracellular matrix, suppression of many intercellular interactions, as well as in promoting angiogenesis. Increased activity of cysteine cathepsins appears in the lungs not only in cancer, but also due to other lung diseases, including cystic fibrosis, COPD, the effects of smoking and others.

In a healthy body there is an enzyme-inhibitor balance, i.e. active cysteine peptidases are controlled by their autogenous inhibitors. In the case of the development of the disease state, in which the activity of cysteine enzymes of peptidases both derived from pathogenic microorganisms and autogenic cathepsins appears, the situation becomes unmanageable. Autogenic inhibitors of these enzymes present in body fluids are autogenic stefins, cystatin and kininogens. However, their amounts are too small to block the activity of cysteine peptidases, whose rapid overexpression accompanies the formation of inflammation associated with disease states. As a result of such changes, the enzyme-inhibitor balance is disturbed, which accelerates the development of the disease and it is not possible to achieve equilibrium in this state again in vivo. Emerging new aggressive enzymes the diseased body is not able to control by autogenic inhibitors and there is no way to supplement their deficiency (Turk V, Stoka V, Turk D. (2008): Cystatins: biochemical and structural properties, and medical relevance. Front Biosci. 13; 5406-5420; Menou A, Duitman J, Crestani B. (2018): The impaired proteases and anti-proteases balance in Idiopathic Pulmonary Fibrosis. Matrix Biol. 68-69: 382-403. Doi: 10.1016/j.matbio 0.2018.03.001; Verma S, Dixit R, Pandey K C. 2016 Cysteine Proteases: Modes of Activation and Future Prospects as Pharmacological Targets. Front Pharmacol. 7, 107-119; and Fear G., Komarnytsky S., Raskin I. (2007): Protease inhibitors and their derivatives as potential drugs. Pharm. & Therap. 113, 354-368).

Literature data indicate that an uncontrolled increase in cathepsin B activity in the brain may be associated with the appearance and development of not only cancerous changes, but also neurodegenerative diseases, including Alzheimer's disease.

Analogous changes—both in the brain and cerebrospinal fluid, were found in neurological diseases. Examples of such diseases are multiple sclerosis (MS) or amyotrophic lateral sclerosis (ALS).

In these diseases, pathogenic cathepsin B is involved in the degradation of motor neurons. Such diseases can be stopped by inhibiting the activity of these enzymes (Haves-Zburof D, Paperna T, Gour-Lavie A, Mandel I, Glass-Marmor L, Miller A. 2011 Cathepsins and their endogenous inhibitors cystatins: expression and modulation in multiple sclerosis. J Cell Mol Med. 15, 2421-2429 and Wilson M E, Boumaza I, Bowser R, 2013 Measurement of cystatin C functional activity in the cerebrospinal fluid of amyotrophic lateral sclerosis and control subjects. Fluids Barriers CNS. 10, 15-23).

It also turned out that the presence of overexpression of cysteine cathepsins in the blood can lead to pathogenic changes in the circulatory system, which result in the development of atherosclerosis and the formation of aneurysms (Lutgens S P., Cleutjens K B., Daemen M J., Heeneman S. 2007 Cathepsin cysteine proteases in cardiovascular disease. FASEB J. 21, 3029-3041 and Zhao C F, Herrington D M. (2016): The function of cathepsins B, D, and X in atherosclerosis. Am J Cardiovasc Dis. 6, 163-170.).

Cysteine cathepsins are also involved in the development of rheumatoid diseases, in which they can also be the target of treatment (Tong B, Wan B, Wei Z, Wang T, Zhao P, Dou Y, Lv Z, Xia Y, Dai Y. 2014 Role of cathepsin B in regulating migration and invasion of fibroblast-like synoviocytes into inflamed tissue from patients with rheumatoid arthritis. Clin Exp Immunol. 177, 586-597).

Studies are also being carried out on cysteine peptidases in various infectious diseases such as malaria and others. It was found that inhibition of the activity of cysteine proteases-falcypain secreted from germs by specific inhibitors may prove to be biocidal for these microorganisms.

It was found that pathogenic cysteine peptidases, which overexpression degrades the human body during illness, are treated in the medical approach as potential targets for new therapeutic directions. This direction applies to both enzymes derived from bacteria, including gingipain, and autogenic cysteine cathepsins associated with the development of cancers and other systemic diseases. As mentioned, in vivo such enzymatic overexpression is controlled by autogenic inhibitors of patients, including from the cystatin superfamily, whose presence and activity is necessary for the precise regulation of physiological processes as well as overexpression in lesions of cysteine peptidase enzymes. (Turk V, Stoka V, Turk D. 2008 Cystatins: biochemical and structural properties, and medical relevance. Front Biosci. 13; 5406-5420).

A good illustration of the issues discussed is the oral cavity and the respiratory system connected to it, creating a local environment in which pathogenic conditions may appear due to the wide range of microorganisms existing there. It is known that even small differences in the quantitative and qualitative balance of the oral flora can lead to disorders that can affect the entire body in the form of pathogenic systemic changes.

Diseases of the oral cavity such as periodontal disease, candidiasis, aggression of herpes viruses, oral cancer, caries and other disease states are accompanied by changes in molecular processes. By following change pathways, new therapeutic methods can be created. Due to the key role of cathepsins in inflammation, this enzyme has become a potential target in the design of therapeutic interventions targeting specific biologically active proteins (Bullon P, Pavillard L E, de la Torre-Torres R. (2018): Inflammasome and oral diseases. Exp Suppl. 108; 153-176. Doi: 10.1007/978-3-319-89390-7_7 and Swanson, K V, Deng, M. & Ting, J P (2019): The NLRP3 inflammasome: molecular activation and regulation to therapeutics. Nat Rev Immunol 19, 477-489 doi: 10.1038/s41577-019-0165-0).

Among the pathogenic bacteria found in the oral cavity, Porphyromonas gingivitalis and its proteolytic enzymes called gingipaines, which are directly responsible for the degradation of periodontal tissues, are best known. These enzymes are extracellular cysteine proteases of this important oral pathogen possessing the strongest virulence factors in this area of the human body. They can catalyze the degradation of a variety of host proteins, helping to avoid host immune responses, deregulate signaling pathways and ultimately lead to tissue destruction.

It is known that the oral cavity is an open microecological environment with over 700 species of microorganisms that under normal conditions maintain a dynamic balance with the host's immune system. When the balance between the host and bacteria is violated, opportunistic pathogens, including broadly understood decision makers of periodontitis, begin to dominate. Changes caused by pathogens cause the destruction of epithelial junctions between the teeth and periodontal tissue, creating a gingival pocket constituting a convenient space for the action of pathogens. The regulation between the host's immune response and pathogens and their periodontal aggression factors has become an important point explaining the mechanism of periodontitis and related systemic diseases (Hočevar K, Potempa J, Turk B. (2018): Host cell-surface proteins as substrates of gingipains, the main proteases of Porphyromonas gingivalis. Biol Chem. 399; 1353-1361. doi: 10.1515/hsz-2018-021.5 and Jia L, Han N, Du J, Guo L, Luo Z, Liu Y. (2019): Pathogenesis of important virulence factors of Porphyromonas gingivalis via toll-like receptors. Front Cell Infect. Microbiol.; 9: 262. Doi: 10.3389/fcimb. 2019.00262).

These enzymes have become significant targets of the immune response in patients and are seen as potential molecular targets in therapeutic approaches to periodontal disease. There is also evidence that P. gingivalis is a well-adapted pathogen of the oral mucosa, known for its participation not only in periodontitis, but also as an important mediator in the development of many diseases such as rheumatoid arthritis and cancers of the mouth or even the entire duct digestive (How K Y, Song K P, Chan K G. (2016): Porphyromonas gingivalis: An overview of periodontopathic pathogen below the gum line. Front Microbiol. 7; 53. doi: 10.3389/fmicb.2016.00053).

It has been shown that the metabolism and ability to colonize epithelial cells and the effect on the immune system of the P. gingivalis host is associated with a very high concentration of cysteine proteinases, gingipain. These enzymes are extracellular products of the main etiological factor in periodontal disease. There is strong evidence that cysteine gingipaines are directly involved in periodontal pocket colonization and lead to destruction of the periodontal support tissue. It has also been established that more than 70% of patients with periodontitis and perimplantitis have pathogens resistant to at least one standard antibiotic, which excludes the possibility of using antibiotics in the treatment of periodontitis (Stone V N, Xu P. (2017): Targeted antimicrobial therapy in the microbiome era Mol. Oral Microbiol. 32; 446-454. Doi: 10.1111/omi.12190).

Relationships between P. gingivalis and gastrointestinal cancers including squamous cell carcinoma of the oral cavity, esophagus in the gastrointestinal tract and pancreatic cancer have also been shown in many clinical and experimental studies because P. gingivalis has a strong relationship with periodontal disease, not only in relation to gastrointestinal tumors, but also the indirect impact of periodontal disease on cancer. One of the most important carcinogenic effects of this bacterium is inhibition of epithelial cell apoptosis, which plays an inherent role in protecting cancer cells. These bacteria can also spread in the blood and even in the brain, which is why they can cause various systemic diseases. Some signaling pathways activated by P. gingivalis involved in cell apoptosis, cancer, as well as in avoiding the immunity and invasion of cancer cells, and in the metabolism of potentially carcinogenic substances caused by this bacterium indicate connections between it and tumors (Zhou Y, Luo G H. Porphyromonas gingivalis and digestive system cancers. World J Clin Cases. 2019 Apr. 6; 7 (7): 819-829. Doi: 10.12998/wjcc.v7.i7.819 and Liu X B, Gao Z Y, Sun C T, Wen H, Gao B, Li S B, Tong Q. (2019): The potential role of P. gingivalis in gastrointestinal cancer: a mini review. Infect Agent Cancer. 14:23. Doi: 10.1186/s13027-019-0239-4).

These bacteria in periodontitis are responsible for 85% of extracellular proteolytic activity at the site of infection caused by this bacterium. For this reason, it is believed that inhibition of gingipain activity should both reduce pathogen survival and alleviate the effects of periodontal inflammation and associated systemic disorders (How, K Y, Song, K P, & Chan, K G (2016). Porphyromonas gingivalis: An Overview of Periodontopathic Pathogen below the Gum Line. Frontiers in microbiology, 7, 53. doi: 10.3389/fmicb.2016.00053).

Periodontosis is one of the manifestations of infectious periodontal tissue diseases. Infections are the cause of chronic gingivitis leading to recession and/or periodontal bone loss (bone defects). In extreme cases, loosening and loss of teeth occurs. This disease affects people and companion animals equally, especially dogs.

Periodontal disease, due to its prevalence, is considered a social disease. These problems are the main cause of tooth loss not only for the elderly, but also for 20-30 years old, and some of their forms are also observed in children. Statistical studies have shown that around 60% of the European Union's population is affected by gum disease to varying degrees, which is the most important cause of tooth loss in patients over 23 years of age, and the proportion of those affected can reach up to 100% of the population in some regions. According to information obtained from WHO, the number of potential patients suffering from oral diseases reaches up to 3 billion worldwide. (Hugoson A., Norderyd O. 2008, Has the prevalence of periodontitis changed during the last 30 years? J Clin Periodontol., 35, 338-345; Susin C, Haas A N, Albandar J M., 2014, Epidemiology and demographics of aggressive periodontitis. Periodontol. 65, 27-452).

Periodontal-related pathologies, according to the American Academy of Periodontology (AAP) classification of 2000, include both gum disease and periodontal disease. Most often these are gingivitis and periodontitis, which arise as a result of an imbalance between periodontal biofilm microbes on the tooth and gum surface and the host's defense mechanisms. The latter are modified by risk factors such as improper oral hygiene, smoking, stress, obesity or diabetes. The occurrence of the disease is the result of the impact of these factors, as well as the genetic effect and the biological phenotype. However, the factor initiating the entire immune-inflammatory reaction responsible for the destruction of periodontal tissues are Gram-negative bacteria such as Porphyromonas gingivalis (P.g), Trepanoma denticola (T.d), Tanerella forsythia (T.f) and Agregatibacter actinomycetemcomitans. Bacteria and toxins stimulate cells that are immunologically competent to secrete inflammatory mediators associated with the immune system, such as PG2, TNF a, IL-1, MMP, which in turn are responsible for the local destruction of tooth-supporting tissues, including connective and bone tissue (Gurav A N. 2014, The association of periodontitis and metabolic syndrome. Dent Res J (Isfahan) 1, 1-10).

As a consequence of these processes in people predisposed to periodontopathy, the response to bacterial inflammation is the activation of macrophages that initiate the secretion of cytokines and T lymphocytes, production of interleukin-17, kappa B ligand receptor receptor (RANKL) or tumor necrosis factor-α. Inflammation and RANKL production induce excessive activation of osteoclasts. This leads to a halting of the bone resorption process, which in turn causes the loss of bone and eventually the tooth (Wade W G. The oral microbiome in health and disease. Pharmacol Res 2013; 69: 137-1434, Racz G Z, Kadar K, Foldes A, Kallo K, Perczel-Kovach K, Keremi B, Nagy A, Varga G. 2014, Immunomodulators and potential therapeutic role of mesenchymal stem cells in periodontitis. J Physiol Pharmacol. 65, 327-339).

There is a clear relationship between pathogenic bacteria in the mouth and so-called red complex, and the level of clinical indicators of periodontal disease such as pocket depth (PD) and bleeding on probing (BOP). In nearly 35% of deep pockets (PD 4 mm), Porphyromonas gingivalis, Tanarella forsythensis and Treponema denticola were detected, while in shallow pockets (PD 3 mm) only in 3.6%. A similar relationship was observed in the case of BOP (+) bleeding, in 35% red complex bacteria were detected, and in the case of BOP (−) only in 3.4% of patients (Komiya Ito A, Ishihara K, Tomita S, Kato T, Yamada S. 2010, Investigation of subgingival profile of periodontopathic bacteria using polymerase chain reaction. Bull Tokyo Dent Coll. 51, 139-1446).

In periodontal disease, there are mainly enzymes derived from pathogenic bacteria that colonize the mouth and autogenic enzymes, produced by the body's own inflammation.

The group of peptide enzymes derived from microorganisms (mainly P. gingivalis) colonizing the periodontal pocket is called gingipain (gingipain-R1, -R2 and -K). They have also been shown to be directly related to periodontal tissue degradation. Investigating their participation in the periodontal tissue degradation processes, three genes were isolated that encode the formation of specific cysteine peptidases that play a role in the processes associated with periodontal disease (de Diego I, Veillard F T, Guevara T, Potempa B, Sztukowska M, Potempa J, Gomis-Rüth F X). 2013, Porphyromonas gingivalis virulence factor gingipain RgpB shows a unique zymogenic mechanism for cysteine peptidases, J Biol Chem. 288, 14287-14296). P. gingivalis is an anaerobic, pathogenic bacterium that leads to many severe periodontal diseases. This pathogen produces numerous virulence factors, fimbria as well as X [Arg-gingipain and B (RgpA and RgpB)] and lysine-X [Lys-gingipain (KGP)]—specific cysteine proteinases are referred to as gingipaines. These factors contribute to varying degrees to bacterial progression and complete destruction of infected periodontal tissues (Veillard F, Sztukowska M, Mizgalska D, Ksiazek M, Houston J, Potempa B, Enghild J J, Thogersen I B, Gomis-Ruth F X, Nguyen K A, Potempa J. 2013, Inhibition of gingipa ins by their profragments as the mechanism protecting Porphyromonas, gingivalis against premature activation of secreted proteases., Biochim Biophys Acta. 1830, 4218-4228).

It is therefore recommended that in the treatment of periodontitis, the activities of the three gingipain known so far be inhibited by their specific inhibitors or specific antibodies (Imamura T. 2003 The role of gingipains in the pathogenesis of periodontal disease. J Periodontol. 74, 111-118).

Similarly, periodontal disease in dogs is one of the most common infectious diseases and a major cause of loss of both teeth and periodontal bone mass. This periodontal destruction and subsequent diseases are called “silent killer” and are one of the most common health problems in dogs over two years old. It is assumed that 85% of dogs after the age of two suffer from gingivitis. Inflammatory changes in the gum area may be reversible when treatment is initiated at the right time. The consequence of not treating this condition is the second stage covering all periodontal tissues. It causes irreversible damage to the ligamentous apparatus of the tooth and the alveolar bone and, consequently, to the loss of teeth.

Destructive changes in the periodontium begin with the formation of tartar forming a rough surface, and the inflammation caused leads to swelling of the gums and the appearance of nagging pain. If no effective prevention or treatment of the inflammatory changes in the gums occurs, the process extends to the remaining periodontal tissues, eventually irreversibly destroying the ligamentous apparatus of the tooth. Teeth loss is caused by inflammation of the gums, which can also lead to abscesses or bleeding.

In dogs, the lack of proper prevention can lead to a drastic decrease in quality of life and its shortening, so it is advisable to include prevention early, even before the changes described above appear. It should be noted that some breeds of dogs, such as Yorkshire Terriers, are particularly susceptible to periodontal disease. It has been shown that the processes accompanying the destruction of the periodontium of domesticated animals (dogs, cats) take place in a susceptible medium in which the action of pathogenic enzymes occurs under analogous conditions as it happens in humans. They differ only in the species causing pathogenic bacteria in gingival pocket fluids. This is probably related to the difference in nutrition of humans and animals, as well as the composition of saliva. Chronic gingivitis creates a risk of bacterial spreading leading to bacteremia. Thus, the immune system is subjected to constant pressure, and within a few years or even months, toxic compounds can cause serious diseases of the liver, kidneys, heart, lungs, and even the entire digestive tract and cause multi-organ failure. Autoimmune diseases of the gums and oropharyngeal mucosa may also appear.

In cats, this disease may also show secondary symptoms due to viral infections such as immunodeficiency, feline leukemia virus and the like. There have also been theories that cats may develop hypersensitivity to tartar. This syndrome is referred to as eosinophilic stomatitis or plasmacytitis.

In humans, gum disease has also been shown to be closely related to cardiovascular disease. It has also been confirmed that bacteria from the gingival pockets often appear in abnormal changes in the heart valves of animals leading to their destruction. These bacteria are identical to those isolated from infected teeth and gums. (Oz H S, Puleo D A. (2011): Animal models for periodontal disease., J Biomed Biotechnol. 1-8: 754857. Doi: 10.1155/2011/754857., Weinberg M A, Bral M. (1999): Laboratory animal models in periodontology, J Clin Periodontol. 26, 335-340.). Changes associated with plaque formation are largely associated with the dog's diet, which indirectly affects his immune system by regulating secreted saliva and its components. Saliva (produced by the salivary glands) contains biologically active proteins responsible for controlling changes in the animal's mouth. Dogs' premolars and molars in dogs have been found to tend to have the most plaque buildup. When tartar forms a rough surface on a tooth, this results in swelling of the gums and pain. Bacteria associated with gingivitis and present in tartar enter the bloodstream and can damage internal organs. This confirms that a lack of proper care for a dog's teeth can lead to a drastic decrease in his quality of life and shortening of life (Harvey C E, Laster L, Shofer F S. 2012, Validation of use of subsets of teeth when applying the total mouth periodontal score (IMPS) system in dogs. J Vet Dent. 29, 222-226). It can be said that most periodontal disease is associated with the development of dental plaque as a result of microbial activity. In this situation, tartar on the border of the gum becomes a direct cause of periodontitis.

Bacteria below the gum line secrete a whole range of chemical compounds (e.g., cytotoxins, proteases, collagenosis, invasins, lactic acid, and acetic and propionic acid) that lead to tissue damage and stimulate the immune system, which can further damage the supporting tissues of the tooth. Then, instead of supporting its own protection system, the immune system escalates inflammation by inducing destruction of periodontal tissues (Harvey C E, Laster L, Shofer F S. 2012, Validation of use of subsets of teeth when applying the total mouth periodontal score (TMPS) system in dogs, J Vet Dent. 29, 222-2266). It has been found that in dogs, just like in humans, specific bacteria play a key role in periodontal degradation. In humans, these changes are caused by so-called bacteria. The red complex, i.e. Porphyromonas gingivalis, Trepanoma denticola, Tanerella forsythia and Agregatibacter actinomycetemcomitans, whereas in dogs mainly P. gingivalis, Prevotella intermedia, Tannerella spp, Peptostreptococcus spp. Particularly important is the role of bacteria from the family of Actada, Hirasawa M. 2000, Expression of trypsin-like activity by the genera, Corynebacterium and Actinomyces in canine periodontitis, J. Med. Microbiol. 49, 621-6257, Stephan B., Greife H A., Pridmore A., Silley P 2008, Activity of pradofloxacin against Porphyromonas and Prevotella spp. Implicated in periodontal disease in dogs: susceptibility test data from a European multicenter study, Antimicrob Agents Chemother. 52, 2149-2155). Direct destructive changes start with enzymes secreted by pathogenic bacteria. The same enzymes are involved in periodontitis in dogs as in analogous changes in humans. It has been well documented that matrix metalloproteinases (MMPs) are involved in periodontal destruction. Therefore, the purpose of understanding the effect of enzymatic degradation of periodontium is to evaluate the effect of MMP and their inhibitors (TIMP; tissue inhibitors of metalloproteinases) in tissues (biopsy) and periodontal fluids in dogs. The presence of MMP-2, MMP-3, MMP-8 and MMP-9 as well as TIMP-1 and TIMP-2 was confirmed in immunohistological studies in both the tissues and gingival pocket fluid. The dog's periodontium has increased expression of the aforementioned metalloproteinases while reducing the expression of their tissue metalloproteinase inhibitors (TIMPs). In summary, the imbalance of MMP-TIMP in dog periodontitis is important for periodontal destruction (Kaiser S M, Thiel C, Kramer M, Raddatz B B, Failing K, Alldinger S. (2015): Immunohistochemical localization and effect of matrix metalloproteinases and their inhibitors on canine spontaneous periodontitis. Vet Rec. 103200. D).

It has also been confirmed that dogs with periodontal inflammation in the gingival pocket fluid also have other active proteolytic enzymes that are used as markers for the advancement of enzymatic changes in periodontitis. They are mainly enzymes that hydrolyze synthetic substrates of both Benzoyl-arginyl-beta-naphthylamide (BANA) and Benzoyl-arginyl-para nitroanilide (BApNA), which are specific substrates for both cysteine and serine peptidases. It was found that by determining the level of activity of the enzymes studied in gingival pocket fluids, it is possible to assess in a simple and fast way the advancement of periodontal inflammation in dogs based on the correlation of their activity and the severity of this disease in the periodontal dog (Kolahi J, Ghalayani P, Varshosaz J. (2006): Systemic toxicity following ingestion of the chlorhexidine gluconate solution: a case report, J Int Acad Periodontol. 8, 45-46).

It is important to indicate the effectiveness of the treatment of inflammatory diseases of dogs gums described below with a synthetic inhibitor of cysteine peptidases and metalloproteinases-chlorhexidine (CHX). This inhibitor has been registered as an antiseptic. It inhibits the activity of metalloproteinases and cysteine peptidases, including gingipain secreted into the gingival pocket fluid by pathogenic bacteria. Inhibition of their activity increases the effectiveness of therapy conducted in periodontal inflammation in dogs. Similarly to humans, drugs with the basic component of CHX up to about 2.0% are used. It can be assumed that this is its maximum concentration due to toxicity (Calogiuri G F., Di Leo E., Trautmann A., Nettis E., Ferrannini A., Vacca A. 2013, Chlorhexidine hypersensitivity: a critical and updated review. 4, 1-712). The use of chlorhexidine in the treatment of periodontal disease can cause serious side effects of strong toxic nature. The combined use of CHX and cystatin from egg white in in vitro studies has been described. (Bhoopathi P G, Kittappa K K., Sanjeev K., Sekar M. (2018): Effect of chlorhexidine and cystatin incorporated adhesives on MMPs and cysteine cathepsin-an in-vitro zymographic analysis. J. Clin. And Diag. Research 12; ZC01-ZC05).

In the treatment of periodontitis, a drug called Ligosan in the form of a periodontal gel is also used. This product contains an antibiotic from the tetracycline group—doxocycline, which has antibacterial activity by inhibiting the synthesis of bacterial enzymes of metalloproteinases. The drug is indicated for the treatment of chronic and aggressive periodontitis in the case of a periodontal pocket equal to or greater than 5 mm and as a complementary method to traditional non-surgical treatment of periodontitis. (Hug M., Lysek D., Koch F., Jung R., Mathes S., Meyer N., Hämmerle Ch., Bröseler F., Pieles, U. (2018): Composition 477 A1 comprising self-assembling peptides for use in treatment of gingivitis, periodontitis and/or periimplantitis—EP 3 284 477 A1

In the treatment of periodontitis, the pharmaceutical company Cortexyme managed to develop a potential drug, KYT-36 and its derivative KYT-41, which inhibit the activity of cysteine gingipain, which are the main virulence factor in periodontosis caused mainly by P. gingivalis. This drug is a synthetic inhibitor that inhibits cysteine gingipain highly selectively and precisely without binding to other enzymes, while the KYT-36 and KYT-41 inhibitors protect better against periodontal changes than tetracycline antibiotics, doxycycline or minocycline and do not show toxicity effects. They are the subject of publications No. US 2016/0096830 A1, US 2017/014468 A1 and WO 2017/201322 A1, as well as JP No. 2010270061 A and JP 4982908 B2 (Kataoka S, Baba A, Suda Y, Takii R, Hashimoto M, Kawakubo T, Asao T, Kadowaki T, Yamamoto K. (2014): A novel, potent dual inhibitor of Arg-gingipains and Lys-gingipain as a promising agent for periodontal disease therapy—FASEB J. 28, 3564-3578 and Guevara T., Rodriguez-Banqueri A., Lasica A M., Ksiazek M., Potempa B A., Potempa J., Gomis-Rüth FX. (2019): Structural determinants of inhibition of gingipain K by KYT-36, a potent, selective, and bioavailable peptidase inhibitor. Sci Rep. 9: 4935. Published online doi: 10.1038/s41598-019-41354-3).

It is currently suggested that the delivery of drugs in a way that allows their local concentration in the tumor microenvironment or inflammation to be locally increased may be significantly better than systemic administration of these drugs. The reason will be to minimize the systemic side effects of the drugs administered. Unfortunately, not all human cancers and inflammation are susceptible to injection of the drug in situ. In the local application, there are also problems with the occurrence of the phenomenon of drug resistance in inflammation, because of which the drugs may degrade and lose their medicinal properties, or a barrier may appear that inhibits their penetration to the focus of the disease.

An interesting alternative to injecting a drug in situ is nebulization using an inhaler. Methods of transferring insulin both through the lungs to the blood and through the olfactory villi to the brain have already been described (Heinemann L. (2011): New ways of insulin delivery. Int J Clin. Pract. 170, 31-46).

The advantage of delivering medicinal substances in aerosols is their non-invasive and effective action at much lower doses as medicinal substances than is required for oral administration or injection. The most important factor in such application of the drug substance is to significantly accelerate its action at the target site. This method allows achieving a high concentration of the medicinal preparation in the whole area infected or at risk of cancer.

Due to the effectiveness and simplicity of use, administration of therapeutic agents in the form of aerosols can definitely outweigh the methods of oral treatment and other parenteral therapies. When this method is used in respiratory therapies, drug arrival sites are closely related to the size of the aerosol mist particles. It is known that these particles settle in various places of this system depending on their size:
5 μm—in the nasopharynx, larynx, trachea and bronchi
2.0-5.0 μm—in the lateral cavities of the nose, other bronchi and bronchioles
0.5-2 μm—in the alveoli
<0.5 μm—are diffused through the respiratory epithelium.

Depending on the planned size and homogeneity of the aerosol droplets, inhalers are selected, including ultrasonic, membrane or other, in which the size of the particles included in the water mist of the aerosol can be designed.

Recently, interest in using inhalation to administer potential therapeutic substances also to the general circulatory system in the treatment of many diseases outside the respiratory tract has also increased. This pathway allows you to control the delivery of the drug to the circulatory system and directly to the brain area. This applies to such diseases as cancer, arteriosclerosis, rheumatoid arthritis, neurodegenerative diseases and many other lesions accompanied by inflammation (Dhanani J., Fraser J F., Chan H K., Rello J., Cohen J., Roberts J A. (2016): Fundamentals of aerosol therapy in critical care. Crit. Care. 20, 269. 1-16 doi: 10.1186/s13054-016-1448-5; Fröhlich E. (2019): Biological obstacles for identifying in vitro-in vivo correlations of orally inhaled formulations. pharmaceutics. 11; 316, 1-19. doi: 10.3390/pharmaceutics11070316 and Cheng Y S. (2014): Mechanisms of pharmaceutical aerosol deposition in the respiratory tract. AAPS PharmSciTech. 15; 630-640.doi: 10.1208/s12249-014-0092-0).

The aerosol, whose components are fog from propylene glycol and glycerol, is approved by the FDA in supplying nicotine in the so-called oral cavity and lungs. e-cigarettes, which are the subject of numerous scientific reports (Jacob M. (2019): Looking Back and Ahead: The Food and Drug Administration's Regulation of the Tobacco Industry and Next-Generation Products. Adv Dent Res. 30; 22-25. doi: 10.1177/0022034519872472, or McNeill A, Driezen P, Hitchman S C, Cummings K M, Fong G T, Borland R. (2019): Indicators of cigarette smoking dependence and relapse in former smokers who vape compared with those who do not: findings from the 2016 International Tobacco Control Four Country Smoking and Vaping Survey. Addiction. 114 Suppl 1: 49-60. Doi: 10.1111/add.14722).

Inflammation—and also lung cancer, is highly predisposed to the use of topically delivered drugs in the form of aerosols that reach them through the intrabronchial space. Aerosol delivery is a promising option in the treatment of lung diseases, and is now a standard approach in the treatment of asthma and chronic obstructive pulmonary disease. In addition to bringing about high local concentration of the applied drug, the advantages of pulmonary aerosol delivery include reduced systemic circulation distribution and painless administration. To date, some drugs used in chemotherapy of lung cancer, such as cytostatics, cytokines, monoclonal antibodies and others have been studied, showing potential options for delivering them in the form of an aerosol. The effectiveness of this type of therapy and its anti-cancer effects have also been confirmed in reducing the side effects that are induced in systemic treatment (Storti C, Le Noci V, Sommariva M, Tagliabue E, Balsari A, Sfondrini L. (2015): Aerosol delivery in the treatment of lung cancer. Curr Cancer Drug Targets. 15; 604-612).

The assumption of new directions in the treatment of diseases accompanied by overexpression of cysteine peptidases is the search for their specific inhibitors showing low toxicity to the human body. It is assumed that inhibiting the activity of these enzymes will achieve a therapeutic effect. Such inhibitors should precisely block the overexpression of pathogenic cysteine peptidases from both autogenic cathepsins and exogenous microbial origin. Cysteine peptidase inhibitors currently at the stage of preclinical studies can be divided into two groups. One of them are inhibitors obtained by chemical synthesis. They are studied mainly in vitro in inhibiting the activity of selected pathogenic enzymes, and exceptionally in vivo. The vast majority of them are eliminated from further research because of their toxicity, which causes the effect of poisoning the body faster than the therapeutic effect. Synthetic inhibitors cause irreversible, covalent modifications of proteins, and their low selectivity is a serious problem for safety and therapeutic efficacy (Siklos M, BenAissa M, Thatcher G R. (2015): Cysteine proteases as therapeutic targets: does selectivity matter? A systematic review of calpain and cathepsin inhibitors. Acta Pharm Sin B. 5, 506-519 and Mitrović A, Kljun J, Sosič I, Uršič M, Meden A, Gobec S, Kos J, Turel I. (2019): Organoruthenated Nitroxoline Derivatives Impair Tumor Cell Invasion through Inhibition of Cathepsin B Activity. Inorg Chem. 58; 12334-12347. doi: 10.1021/acs.inorgchem.9b01882).

However, some of these synthetic inhibitors have been approved for treatment despite significant toxicity. The reason was the need to effectively and precisely block the activity of pathogenic enzymes. Therapeutic efficacy is higher than the side effects of using such a drug. Examples are chlorhexidine in the treatment of periodontitis (Scaffa P M, Vidal C M, Barros N, Gesteira T F, Carmona A K, Breschi L, Pashley D H, Tjaderhane L, Tersariol I L, Nascimento F D, Carrilho M R. 2012 Chlorhexidine inhibits the activity of dental cysteine catheps J Dent Res. 91, 420-425).

Inflammatory changes in the gums, periodontal disease have a large impact on the patient's overall health and even life-threatening condition. Therefore, CHX was approved for clinical use despite its toxicity due to the danger of periodontitis causing other diseases such as circulatory, rheumatoid, alzheimer's, diabetes, lung diseases or pregnancy complications (Bui F Q, Almeida-da-Silva C L C, Huynh B, Trinh A, Liu J, Woodward J, Asadi H, Ojcius D M. (2019): Association between periodontal pathogens and systemic disease. Biomed J. 42; 27-35).

Chlorhexidine as the most effective known drug used in the treatment of periodontitis is a de facto inhibitor of cysteine gingipain, inhibiting the activity of these enzymes. Effective blocking of the activity of this key virulence factor produced by P. gingivitalis bacteria—cysteine gingipain, decided to allow this drug to protect the gums and oral cavity against gingivitis, paradontosis (Cronan C A, Potempa J, Travis J, Mayo J A. (2006): Inhibition of Porphyromonas gingivalis proteinases (gingipains) by chlorhexidine: synergistic effect of Zn (II). Oral Microbiol Immunol. 21, 212-217).

Regardless of the use of synthetic inhibitors in the treatment, attempts are being made to obtain natural inhibitors—cystatin from biological sources. The methods of obtaining them previously known were carried out mainly in laboratory conditions, using complicated and expensive isolation techniques. These studies are still in the initial stages of development mainly due to the efficiency of their acquisition and their stability (Turk V., Brzin J.: Process for the isolation of chicken egg white cystatine, antiviral agents containing it and its use as viral protease inhibitor. Patent Application No. EP 0188262 A2).

European publication no. EP 2 511 288 (application no. EP 12163789.6) discloses a method for isolating cystatin from natural, human-friendly raw materials. They show the ability to inhibit the activity of pathogenic cysteine peptidases (autogenic and bacterial). It turned out, however, that under native conditions they are low stable and lose their activity, including due to dimerization. However, there was a chance to store them in glycerol or polyglycols. In such conditions they maintained their activity even for many months (Siewiński M., Czarnecki J., Jujeczka S., Seliga J., Żochowski A. 2012: The method of obtaining inhibitors of cysteine peptidases with an electrophoretic purity from biological materials” EP 2 511 288).

Polish Patent Application No. PL 428931, which is the basis for the priority claimed for the present invention, discloses the use of hen egg cystatin in inhibiting cysteine peptidases, including gingipain, in the manufacture of a medicament useful in the prevention and treatment of periodontitis in humans and animals. This disease affects people and companion animals equally, especially dogs. The low stability of the cystatin used was leveled by the addition of glycerol or glycols. In humans, this inhibitor blocks about 85% of all proteolytic activity in the gingival pocket fluid, which is probably the activity associated with gingipaines secreted by the pathogen P. gingivitalis. In dogs, egg white cystatin blocks about 90% of total proteolytic activity. This result suggests that in gingival pocket fluid in people with periodontitis, about 15% of proteolytic activity and about 10% of activity in dogs is associated with the participation of other proteolytic enzymes than cysteine peptidases (Siewiński M., Rapak A., Raźniewski J. (2019): Use of cysteine gingipain inhibitors for the production of a therapeutic agent useful in the prevention and treatment of periodontitis in humans and companion animals, and a therapeutic agent useful in the prevention and treatment of periodontitis in humans and companion animals, containing cysteine gingipain inhibitors. PL 428931).

For the isolation of cysteine peptidase inhibitors, plants have also proved to be a preferred raw material. They are cheap, easily available and mostly safe for the human body. Plant cystatins are naturally occurring inhibitors that prevent proteolytic changes that can cause cysteine proteases in plants. Their protective effect in plants against various threats has been relatively well characterized. Cystatin in plants protect them against diseases associated with the participation of parasites, as well as against other factors during their development from the moment of germination throughout the growth. Independent of plants living in natural conditions, there are also transgenic varieties that have been modified to protect them from environmental stress. Transgenic plants can be a source in the production of recombinant proteins, which, however, require additional safety controls (van Wyk S G, Kunert K J, Cullis C A, Pillay P, Makgopa M E, Schlüter U, Vorster B J. (2016): Review: The future of cystatin engineering. Plant Sci. 246: 119-127. doi: 10.1016/j.plantsci.2016.02.016).

Natural products of plant origin are studied extensively, including their therapeutic role in regulating interactions between microorganisms. One attractive therapeutic feature is that bioactive compounds from plants appear safe and should not cause toxicity to human cells. However, comprehensive toxicity testing of these compounds is still considered necessary (How K Y, Song K P, Chan K G. (2016): Porphyromonas gingivalis: An overview of periodontopathic pathogen below the gum line. Front Microbiol. 7; 53. doi: 10.3389/fmicb.2016.00053).

The purpose of the present invention is to provide non-toxic, specific cysteine inhibitors of peptidases, stable at ambient temperature, isolated from natural raw materials, how to extract them from available organic natural raw materials, and to build a foundation for a new direction in the treatment of diseases associated with the overexpression of pathogenic cysteine peptidase enzymes, based on the use of the indicated inhibitors in human and veterinary medicine, enabling the selective inhibition of overexpression of pathogenic enzymes directly in affected tissues.

In another aspect, it is an object of the present invention to solve the problems known in the art and resulting from the development of gingivitis and to provide a therapeutic agent useful in the prevention and treatment of periodontal disease, counteracting the undesirable effects of periodontitis in humans and companion animals, especially dogs.

It has now surprisingly been found that the above goals can be achieved by identifying low molecular weight cysteine inhibitors of peptidases of natural origin and developing a method for isolating low molecular weight cysteine inhibitors of peptidases from natural organic materials.

In addition, the current studies have unexpectedly confirmed the possibility of administering these inhibitors to patients in need of this by nebulization using an inhaler. There is also the possibility of transmitting currently isolated peptide inhibitors via the lungs to the blood, and with it to all organs that the blood reaches. It is also possible to deliver these inhibitors with blood or via epithelial cells in the olfactory villi to the brain area. The use of indicated inhibitors is contemplated to control overexpression of cysteine peptidases in the space involving the sinuses, and—after crossing the blood/brain barrier, also the brain area. In this way, the inhibitors of the invention will reach diseased sites colonized by pathogenic microorganisms or mutated cells, such as cancer cells, for example. The administration of inhibitors is aimed at blocking the overexpression of cysteine peptidases initiating pathogenic changes in the human body. In this regard, the prototype of the present invention are methods of transferring insulin both through the lungs to the blood and through the olfactory villi to the brain.

The subject of the invention is defined in detail in the attached independent claims. Preferred embodiments are set out in the dependent claims.

The invention includes new low molecular weight cysteine peptidase inhibitors of natural origin, which are several-amino acid oligopeptides with a molecular weight below 3 kDa, which are additionally free of salts with a molecular weight below 700 Da. The source of the new low molecular weight inhibitors according to the invention are the following natural organic materials: egg white, casein, milk, knotweed—especially of the species Fallopia japonica, Houtt, mistletoe, soybean, pineapple, rice, potatoes and other substances of natural origin.

The invention also includes cystatin isolated from knotweed, especially the Fallopia japonica, Houtt species, stable in a glycerol- and other glycol-free environment. The invention also includes cystatin isolated from knotweed, especially the Fallopia japonica, Houtt species, stable in a glycerol- and other glycol-free environment.

In addition, the invention includes a method for isolating low molecular weight cysteine inhibitors of peptidases of the invention.

New low molecular weight cysteine peptidase inhibitors of natural origin, which are several-amino acid oligopeptides with a molecular weight below 3 kDa, and cystatin extracted from knotweed, especially of the species Fallopia japonica, Houtt, as defined above, in accordance with the present invention find (first) medical use in human and veterinary medicine. Their use for the production of medical agents useful in the treatment and prevention of periodontitis in human and veterinary medicine, as well as for the production of medical agents useful in the treatment and prevention of malignant tumors accompanied by cathepsins.

Preferably, the medicaments containing the inhibitors of the invention are adapted to be administered as aerosols directly into the entire area of the mouth, larynx, esophagus, bronchi and lung area, as well as the area of the sinuses and the area called the blood/brain barrier with the possibility of reaching the brain and through lungs with blood circulation to other disease centers throughout the body.

Surprisingly, it turned out that cystatin separated from the new, low-molecular cysteine inhibitors of the peptidases of the invention, isolated from the knotweed protein mass, especially the Fallopia japonica, Houtt species, is incomparably more durable than cystatin isolated in the same way from egg protein and does not require glycerin stabilization

The low molecular weight cysteine peptidase inhibitors of the invention are in fact an unidentified by-product of the process of extracting cystatin from egg protein and from plant materials such as knotweed, especially the species Fallopia japonica, Houtt, mistletoe, pineapple, potatoes and other existing sources of natural cystatines of plant origin and other substances of origin natural.

In the following detailed description of the invention, all practical aspects of the present invention are presented and discussed, including numerous embodiments

### DETAILED DESCRIPTION OF THE INVENTION

In the course of research on the invention, conducting the isolation of cystatins from various biological raw materials, it was surprisingly found that these cystatin inhibitors of cysteine peptidases, with a molecular weight of about 13 kDa are accompanied by a fraction of hitherto undescribed short oligopeptides composed of several-amino acids, showing high stability and high inhibitory activity relative to pathogenic cysteine peptidases, both autogenic and microbial. The present invention includes a method for their isolation. These low molecular weight inhibitors are isolated according to the subsequent steps of the process for purifying cystatin from egg protein and selected plants, known from patent EP 2 511 288. These peptides appear together with cystatins in an eluate from an affinity chromatography column filled with Sepharose 4B previously papain was immobilized. In the next step, the new low-molecular cysteine peptidase inhibitors of the invention, which are short oligopeptides composed of several-amino acids, are separated from cystatin.

As a result of the research, it was found that these low molecular weight cysteine peptidase inhibitors of the invention inhibit active cysteine peptidases both in pure form (such as cathepsin B) and cysteine cathepsins found in tumor tissues or atherosclerotic arteries.

The low molecular weight cysteine peptidase inhibitors of the invention also inhibited the activity of cysteine gingipain in gingival pocket fluids of patients with periodontitis.

The research included:
isolation of cystatins from natural raw materials not toxic to the human body, and
isolation from the same natural raw materials, non-toxic to the human body, low-molecular cysteine inhibitors of peptidases that inhibit pathogenic cysteine peptidases such as: autogenic cathepsins, including cathepsin B and enzymes of bacterial origin, including gingipain, as well as
identification of low-molecular cysteine inhibitors of peptidases according to the invention (with possible determination of the sequence of isolated short peptides).

Further research determined the ability of isolated inhibitors to inhibit pathogenic cysteine peptidases in:
Native cathepsin B,
homogenates of lung cancer tissues,
gastrointestinal cancer homogenates,
tissue homogenates after thyroid surgery,
vein tissue homogenates collected from patients with atherosclerosis,
gingival pocket fluids collected from patients with periodontitis (gingapain).

Native cathepsin B is considered a promoter of diseases such as cancer, dementia (Alzheimer's and others), neurological diseases (multiple sclerosis—MS, amyotrophic lateral—ALS) and a number of other diseases.

Isolation of low molecular weight cysteine peptidase inhibitors of the invention, which are oligopeptides: Oligopeptides, which are low-molecular inhibitors of cysteine peptidases, covering several-amino acids, have been isolated from egg protein as well as plant raw materials (knotweed, sugar cane, sugar beet, pineapple, mistletoe). Edible products that could be food ingredients were used as isolation raw materials, which indicated a lack of toxicity to human organisms. These raw materials can be described as “friendly to the human body”.

Autogenous cysteine peptidase inhibitors—such as kininogen, cystatin and currently identified several-amino acid peptides, can also be isolated from the urine of cancer patients, from placenta or from cancerous tissues that were removed from the patient's body during surgery. Autogenous inhibitors will be useful for use in personified therapies.

The method of isolating low-molecular cysteine inhibitors of the peptidases of the invention, which are oligopeptides composed of several-amino acids, capable of inhibiting cysteine peptidases: cathepsins and gingipain, from biological raw materials, according to the invention is the next step in the method of obtaining cystatin, known from patent EP 2 511 288. Preparation of low molecular weight peptide inhibitors differs from the isolation of cystatins in that they do not add glycerol or other cystatin stabilizing compounds to the solutions collected after the column with a Papain Sepharose 4B bed. Selected natural organic raw material—egg protein or milk, or fresh or dried plants, which are a source of cysteine peptidase inhibitors, mixed with water and homogenized, then acidified to pH=2.0 or adjusted to pH=10, heated for 20 minutes in 90° C., after which the whole cools down and neutralizes. Then it freezes at −20° C. for 24 hours, then thaws and spins 30 minutes at 25,000 rpm. 10% by volume of 0.01M phosphate buffer at pH 6.0 is added to the supernatant, the whole is filtered on paper and applied to a column filled with Sepharose 4B with papain immobilized on it. After applying the biological material, the column is washed first with 0.05% NaCl and then with water until the protein in the eluent has disappeared. Then 0.01% NaHCO3, with pH adjusted to 11.0 using 0.1 M NaOH is applied to the column. When washing the column with buffer at pH=11, the presence of protein in the eluent is controlled, preferably using a fraction collector. After collecting the protein-containing eluent, the whole is neutralized and, if necessary, filtered or centrifuged so that the solution is clear. It contains cystatin and new low-molecular cysteine peptidase inhibitors of the invention, which are several-amino acid peptides having affinity for cysteine peptidases, for example immobilized papain, i.e. molecules with cysteine peptidase inhibitors.

The protein fractions are combined and the whole is subjected to separation on a membrane permeable compounds of a certain size. Because at this stage the interest is focused on the isolation of peptides and not cystatins, the combined protein fractions were not stabilized with glycerol or other glycols. The main reason for not using a stabilizer is the high probability of blocking the filters used in the separation stage. The combined protein fractions (eluate from the affinity chromatography column) are filtered as quickly as possible using membrane permeable to 3 kDa molecules, and then the low molecular filtrate is desalted on membrane permeable to 700 Da. The filtrate contains low molecular weight inhibitors, while cystatin does not pass through the filters. The resulting low molecular weight oligopeptides are able to inhibit cysteine peptidase enzymes such as autogenic cathepsins or bacterial enzymes. The low molecular weight cysteine peptidase inhibitors of the invention thus obtained are stored either in a refrigerator (+4° C.) or in a freezer.

In current studies, it has surprisingly been found that cystatin obtained—after separating the low-molecular cysteine inhibitors of the peptidases of the invention from knotweed (Fallopia japonica, Houtt.) Retained high separation stability despite the absence of a stabilizer in the form of glycerol or other glycols.

When using inhalers, the solutions with the inhibitors according to the invention are nebulized and in the form of an aerosol mist delivered directly to the entire oral cavity, sinuses and respiratory system, i.e. nose, throat, larynx, trachea, bronchi and lungs, including to the affected areas.

Inhibitors according to the invention are isolated from natural raw materials and at the same time “friendly” to the human body, obtained on the basis of the solutions according to the application No. EP 2 511 288 and the method according to the invention first disclosed in the application No. PL 431519, constituting the basis of the conventional priority claimed for the present invention.

Using active and non-toxic inhibitors of cysteine peptidases administered in aerosols, the reality of a new direction in the treatment of diseases associated with the selective inhibition of overexpression of pathogenic enzymes, referred to as “in situ inhalation inhibitortherapy”, was confirmed.

Inhibitors according to the invention are natural cysteine peptidase inhibitors such as cystatin, low molecular weight peptides or other fractions isolated from the knotweed Fallopia japonica, Houtt by the method of the invention. The invention also envisages the possibility of replacing them with analogous inhibitors obtained from other plants or biological materials derived from vertebrates: milk, egg white, placental and human and animal fetal waters, blood, urine, tissues and the like, which will be isolated according to the method of the invention. Several-amino acid peptides can also be replaced by their analogues obtained by chemical synthesis.

Similarly, the inhibitors of the invention find medical use in the selective blocking of exogenous cysteine peptidases originating in pathogenic cells in animal organisms, including domesticated ones such as dogs and cats, including others (Siewiński M., Czarnecki J., Jujeczka S., Seliga J., Żochowski A. (2012): The method of obtaining inhibitors of cysteine peptidases with an electrophoretic purity from biological materials. EP 2 511 288, as well as Siewiński M., Rapak A. (2019): Inhibitors of cysteine peptidases, of natural origin and a method for isolating low-molecular cysteine inhibitors of peptidases from natural organic materials. Polish Patent Application No. PL 431519).

As mentioned, administration of medicinal substances in aerosols—also the inhibitors according to the invention, allows achieving a high concentration of the medicinal preparation in the whole oral cavity, larynx and lungs. The nebulization preparations of the natural cysteine inhibitors of the peptidases of the invention are suitable for use in the treatment of various upper and lower respiratory tract diseases. These preparations, meeting additional criteria, find use in blocking overexpression of cysteine peptidases throughout the body, including the brain.

Problems related to the stability of cystatin isolated from egg white prompted the search for other raw materials from which more stable cystatin can be obtained, which would also guarantee the biological safety of the raw material.

The method of isolating the inhibitors of the invention leads to the production of cystatins of plant origin, and in parallel with cystatins, it is possible to isolate from natural raw materials a system of 3-4 (several) amino acid peptides with the properties of cysteine peptidase inhibitors. These peptides have high stability. The research was conducted in two ways:
Isolation of cystatin from natural raw materials (non-toxic to the human body), and
Isolation of several-amino acid peptides that inhibit pathogenic cysteine peptidases, such as: autogenic cathepsins, including cathepsin B and enzymes of bacterial origin, including gingipain, from the same raw materials.

Cathepsin B is considered a promoter of diseases such as cancer, dementia (Alzheimer's disease and others), neurological (multiple sclerosis—MS, amyotrophic lateral—ALS) and others.

By the method of the invention, several-amino acid inhibitors were obtained from both egg white and selected plants, such as knotweed Fallopia japonica, Houtt, mistletoe, sugar cane, sugar beet, pineapple and several other plant materials.

The method of the invention also allows the isolation of the indicated inhibitors from other plants and from raw materials derived from vertebrates, such as urine, placenta, fetal water, blood and others. Surprisingly, it has also been found that peptide low molecular weight inhibitors from egg protein, unlike cystatins from this raw material, maintain high stability. These inhibitors inhibit the activity of both papain and cathepsin B, gingipain in gingival pocket fluids, as well as cysteine cathepsins in tumor homogenates.

Cystatin obtained from plant materials, including mainly from the knotweed Fallopia japonica, Houtt, works in a similar way. Already in the early stages of research, it was shown that in fluids from the gingival pockets, cystatin and low-molecular peptides inhibit activity in about 85% of total proteolytic activity, i.e. to a similar extent as the activity of gingipain from P. gingivitalis. In contrast, in lung and colorectal cancer tissues, low molecular weight inhibitors from knotweed and egg protein as well as cystatin from knotweed inhibited proteolytic activity by about 65%.

Based on these studies and observations, a proposal was made for the therapeutic use of obtained stable plant cystatins and several-amino acid inhibitors that act precisely and selectively block the activity of cysteine peptidases—both secreted by pathogenic microorganisms and autogenic cysteine cathepsins, since it is known that such cathepsins initiate many pathogenic changes in the area of the whole human body (Siewiński M., Rapak A., Raźniewski J., Nieradko Ł. (2019): Inhibitors of cysteine peptidases, of natural origin and a way of isolating low-molecular cysteine inhibitors of peptidases from natural organic materials. Polish Patent Application No. PL 431519).

It has now been found that the nebulization process has no effect on the activity of cysteine inhibitors of peptidases isolated from knotweed Fallopia japonica, Houtt. This allowed the development of a new route of administration of non-toxic cysteine inhibitors of peptidases from natural raw materials in the form of aerosols produced in ultrasonic inhalers. In this form, biologically stable and practically non-toxic peptides can reach the entire oral cavity and upper and lower respiratory tract in aerosols in the human body. In these locations, they are able to inhibit the activity of both papain, native cathepsin B, cysteine cathepsins in lung cancer homogenate, as well as gingipain with P. gingivitalis in gingival pockets of patients with periodontitis (periodontitis).

Determination of Cysteine Peptidase Activity

In gingival pocket fluids, cysteine gingipain activity was determined as the amount of p-nitroaniline released from BApNA (Benzoyl-DL Arginyl-para-nitroanilide) substrates, or Z-Arg-Arg-pNA (Benzol-DLArg-Arg-β-p-nitroanilide).

The activity of cysteine gingipain in gingival pocket fluids was determined using BApNA, while the activity of cysteine cathepsins in tumor homogenates and arteries of people with atherosclerosis and native cathepsin B with the chromogenic substrate Z-Arg-Arg-p-NA.

The amount of cysteine peptidases that was used as enzymatic activity was that led to the degradation of the substrates used and release of micromoles of p-nitroaniline from them within 1 minute at 37° C. The activity of these enzymes was inhibited by cysteine peptidase inhibitors, which were: knotweed cystatin (Fallopia japonica, Houtt.) And new low-molecular cysteine peptidase inhibitors of the invention (several-amino acid peptides) from egg and knotweed protein. Cystatin from knotweed was strictly isolated based on the method described in patent No. EP 2 511 288, while the new low molecular weight cysteine peptidase inhibitors of the invention based on the method of the invention described in the present application.

One inhibitory unit is the number of inhibitors that is able to inhibit 1 unit of enzyme activity (Mailhot J M, Potempa J, Stein S H, Travis J, Sterrett J D, Hanes P J, Russell C M. (1998): A relationship between proteinase activity and clinical parameters in the treatment of periodontal disease. J Clin Periodontol. 25, 578-584 and Hasnain S, Hirama T, Huber C P, Mason P, Mort J S. (1993): Characterization of Cathepsin BB specificity by site-directed mutagenesis. Importance of GIu245 in the S2-P2 specificity for arginine and its role in transition state stabilization. J Biol Chem. 268, 235-240).

Inhibition of Cysteine Gingipain Activity

Inhibition was performed in 10 fluid samples from gingival pockets taken from patients diagnosed with periodontitis. Cysteine gingipain activity was inhibited by knotweed cystatin and isolated new low molecular weight cysteine peptidase inhibitors of the invention. The results obtained are summarized in Tables 1-6 below.

TABLE 1
Inhibition of cysteine gingipain with cystatin from knotweed

| | Activity before | Activity after | % | % |
|---|---|---|---|---|
| Sample | adding inhibitor | adding inhibitor | remaining | inhibited |
| number | [U/ml] | [U/ml] | activity | activity |
| 1 | 2.12 | 0.31 | 15 | 85 |
| 2 | 1.83 | 0.28 | 15 | 85 |
| 3 | 1.15 | 0.16 | 14 | 86 |
| 4 | 1.65 | 0.35 | 21 | 79 |
| 5 | 0.93 | 0.18 | 19 | 81 |
| 6 | 2.38 | 0.45 | 19 | 81 |
| 7 | 0.78 | 0.23 | 21 | 79 |
| 8 | 1.96 | 0.30 | 15 | 85 |
| 9 | 1.12 | 0.14 | 13 | 87 |
| 10 | 0.93 | 0.18 | 19 | 81 |
| | | Average | 17.9 ± 4.7 | 83.1 ± 4.7 |

TABLE 2
Inhibition of cysteine gingipain low molecular
weight inhibitors from knotweed

| | Activity before | Activity after | % | % |
|---|---|---|---|---|
| Sample | adding inhibitor | adding inhibitor | remaining | inhibited |
| number | [U/ml] | [U/ml] | activity | activity |
| 1 | 2.12 | 0.34 | 16 | 84 |
| 2 | 1.83 | 0.21 | 12 | 88 |
| 3 | 1.15 | 0.14 | 12 | 88 |
| 4 | 1.65 | 0.39 | 24 | 76 |
| 5 | 0.93 | 0.14 | 15 | 85 |
| 6 | 2.38 | 0.48 | 20 | 80 |
| 7 | 0.78 | 0.19 | 25 | 75 |
| 8 | 1.98 | 0.32 | 16 | 84 |
| 9 | 1.12 | 0.17 | 15 | 85 |
| 10 | 0.93 | 0.12 | 13 | 87 |
| | | Average | 16.6 ± 4.6 | 83.6 ± 4.6 |

TABLE 3
Inhibition of cysteine gingipain low molecular
weight inhibitors from egg protein

| | Activity before | Activity after | % | % |
|---|---|---|---|---|
| Sample | adding inhibitor | adding inhibitor | remaining | inhibited |
| number | [U/ml] | [U/ml] | activity | activity |
| 1 | 2.12 | 0.48 | 23 | 77 |
| 2 | 1.83 | 0.33 | 18 | 82 |
| 3 | 1.15 | 0.19 | 17 | 83 |
| 4 | 1.65 | 0.40 | 24 | 76 |
| 5 | 0.93 | 0.19 | 21 | 79 |
| 6 | 2.38 | 0.51 | 22 | 78 |
| 7 | 0.78 | 0.21 | 27 | 73 |
| 8 | 1.96 | 0.34 | 17 | 83 |
| 9 | 1.12 | 0.19 | 17 | 83 |
| 10 | 0.93 | 0.15 | 16 | 84 |
| | | Average | 20.0 ± 3.7 | 80.0 ± 3.7 |

TABLE 4
Inhibition of cysteine cathepsins in lung cancer tissue:

| | The | Activity before | Activity after | | |
|---|---|---|---|---|---|
| | inhibitor | adding | adding | % | % |
| | amount | inhibitor | inhibitor | remaining | inhibited |
| Inhibitor | [μl] | [U/ml] | [U/ml] | activity | activity |
| 1 | 0 | 0.83 | 0.83 | 100 | 0 |
| | 20 | | 0.68 | 82 | 18 |
| | 50 | | 0.56 | 67 | 33 |
| | 100 | | 0.47 | 57 | 43 |
| | 300 | | 0.28 | 36 | 64 |
| 2 | 0 | 0.83 | 0.83 | 100 | 0 |
| | 20 | | 0.58 | 70 | 30 |
| | 50 | | 0.43 | 52 | 48 |
| | 100 | | 0.28 | 33 | 67 |
| | 300 | | 0.15 | 18 | 82 |
| 3 | 0 | 0.83 | 0.83 | 100 | 0 |
| | 20 | | 0.62 | 75 | 25 |
| | 50 | | 0.48 | 58 | 42 |
| | 100 | | 0.35 | 42 | 58 |
| | 300 | | 0.22 | 26 | 74 |

1-low molecular weight egg white inhibitors
2-low molecular weight knotweed inhibitors
3-knotweed cystatin

TABLE 5
Inhibition of cysteine cathepsins in colorectal cancer tissue:

| | The | Activity | Activity | | |
|---|---|---|---|---|---|
| | inhibitor | before adding | after adding | % | % |
| | amount | inhibitor | inhibitor | remaining | inhibited |
| Inhibitor | [μ] | [U/ml] | [U/ml] | activity | activity |
| 1 | 0 | 1.04 | 1.04 | 0 | 100 |
| | 20 | | 0.91 | 88 | 12 |
| | 50 | | 0.78 | 75 | 25 |
| | 100 | | 0.45 | 43 | 57 |
| | 300 | | 0.27 | 37 | 63 |
| 2 | 0 | 1.04 | 1.04 | 0 | 100 |
| | 20 | | 0.86 | 83 | 17 |
| | 50 | | 0.68 | 65 | 35 |
| | 100 | | 0.41 | 59 | 61 |
| | 300 | | 0.21 | 20 | 80 |
| 3 | 0 | 1.04 | 1.04 | 0 | 100 |
| | 20 | | 0.88 | 85 | 15 |
| | 50 | | 0.70 | 67 | 33 |
| | 100 | | 0.43 | 41 | 59 |
| | 300 | | 0.23 | 22 | 78 |

1-low molecular weight egg white inhibitors
2-low molecular weight knotweed inhibitors
3-knotweed cystatin

TABLE 6
Inhibition of cysteine gingipain with cystatin
from egg white

| | | CP | 100-F/E*100 | |
|---|---|---|---|---|
| | | activity | Remaining CP | |
| | Cysteine | after | activity after | E/F*100 |
| | peptidases | adding | inhibition with | inhibited |
| Sample | activity | cystatin | cystatin | activity |
| number | [U/ml] | [U/ml] | % | % |
| 1 | 2.57 | 0.47 | 81.71 | 18.29 |
| 2 | 2.17 | 0.61 | 71.89 | 28.11 |
| 3 | 10.95 | 1.16 | 89.41 | 10.59 |
| 4 | 9.72 | 0.98 | 89.92 | 10.08 |
| 5 | 4.17 | 1.12 | 73.14 | 26.86 |
| 6 | 3.96 | 0.75 | 81.06 | 18.94 |
| 7 | 3.53 | 0.34 | 90.37 | 9.63 |
| 8 | 8.29 | 0.47 | 94.33 | 5.67 |
| 9 | 7.89 | 0.87 | 88.97 | 11.03 |
| 10 | 8.25 | 1.22 | 85.21 | 14.79 |
| 11 | 9.18 | 0.98 | 89.32 | 10.68 |
| 12 | 7.76 | 1.11 | 85.70 | 14.30 |
| 13 | 5.23 | 0.47 | 91.01 | 8.99 |
| 14 | 14.84 | 2.19 | 85.24 | 14.76 |
| 15 | 9.77 | 1.12 | 88.54 | 11.46 |
| 16 | 11.68 | 2.52 | 78.43 | 21.57 |
| 17 | 7.93 | 1.97 | 75.16 | 24.84 |
| 18 | 16.51 | 3.96 | 76.00 | 24.00 |
| 19 | 9.82 | 2.18 | 73.80 | 22.20 |
| 20 | 12.71 | 3.11 | 75.53 | 24.47 |
| 21 | 9.23 | 1.28 | 83.13 | 13.87 |
| 22 | 13.63 | 2.74 | 79.90 | 20.10 |
| 23 | 10.28 | 1.73 | 83.17 | 16.83 |
| | | Average: | 83.08 ± 6.63 | 15.89 ± 6.28 |

The method of isolating the low-molecular cysteine inhibitors of the peptidases of the invention described above gives positive results using the protein mass of knotweed (Fallopia japonica, Houtt), egg protein, milk protein and such plants as sugar cane, sugar beet, pineapple, mistletoe. As indicated, the method of isolating low-molecular peptides with the properties of cysteine inhibitors of the peptidases of the invention is a continuation of the isolation of cystatin from natural raw materials, preferably food presented in patent No. EP 2 511 288. It has been found that low-molecular inhibitors occur in milk protein and mistletoe, pineapple and sugar cane and probably other natural resources. Using the presented method of isolating cysteine inhibitors of peptidases with a molecular weight of about 13 kDa, we also noticed the presence of short peptides. Their isolation proved to be possible using affinity chromatography on a Sepharose 4B-papain. After applying a solution containing water-soluble protein components (eggs and milk) or supernatants from homogenates of selected plants to the column, specific binding to the cystatin deposit and associated low-molecular peptides occurs. Cystatin and peptides have an affinity for immobilized papain strong enough that they are not washed away when washed with water and 0.1 M NaCl, suggesting that they are inhibitors and not molecules that bind to papain non-specifically. Both compounds are released after washing the column with a solution of alkaline or acidic pH. Separation of cystatin from low molecular weight peptide inhibitors was achieved by filtration using 3 kDa filters. Most cystatins from natural raw materials isolated by this method remained stable except for cystatin from chicken egg protein, which required stabilizers such as glycerol or polyglycols to maintain activity. Low-molecular peptides behave similarly, which show stable activity of cysteine peptidase inhibitors even after storage for several months in the refrigerator (about 4° C.). Based on the described method, it is also possible to isolate inhibitors from cancer tissues obtained intraoperatively or from the urine of patients. Isolations of such autogenous inhibitors for patients can be used in experimental personalized therapies.

Periodontitis

Inflammatory changes in the gums, periodontal disease have a large impact on the patient's overall health and even life-threatening condition. Therefore, CHX was approved for clinical use despite its toxicity due to the danger of periodontitis causing other diseases such as circulatory, rheumatoid, alzheimer's, diabetes, lung diseases or pregnancy complications (Bui F Q, Almeida-da-Silva C L C, Huynh B, Trinh A, Liu J, Woodward J, Asadi H, Ojcius D M. (2019): Association between periodontal pathogens and systemic disease. Biomed J. 42; 27-35).

The ability of tetracyclines to inhibit pathogenic gingipain has meant that this family's antibiotics find use in the treatment of periodontitis. Chemical modification of tetracyclines is also introduced to eliminate their antibiotic activity while maintaining (or even strengthening) their anti-collagenase inhibitory properties. (Golub L M, Elburki M S, Walker C, Ryan M, Sorsa T, Tenenbaum H, Goldberg M, Wolff M, Gu Y. (2016): Non-antibacterial tetracycline formulations: host-modulators in the treatment of periodontitis and relevant systemic diseases Int Dent J. 66 127-135).

In Polish application No. PL 428931 of Feb. 15, 2019, it was proposed to use cystatin isolated from egg white in the treatment of periodontitis. It was found that this inhibitor inhibited proteolytic activity in gingival pocket fluids in about 85%. (Siewiński M., Rapak A., Raźniewski J., 2019, The use of cysteine gingipain inhibitors for the production of a therapeutic agent useful in the prevention and treatment of periodontitis in humans and companion animals, and a therapeutic agent useful in the prevention and treatment of periodontitis in humans and companion animals, containing cysteine gingipain inhibitors).

Similar results of inhibition of proteolytic activity in gingival pocket fluids were also found during research on the present invention. It turns out that not only cystatin from egg protein, but also from knotweed and several-amino acid oligopeptides from both knotweed and egg protein lead to inhibition of proteolytic activity by 85% compared to total activity. These inhibitors, like cystatin from egg white, were unable to inhibit more proteolytic activity even when excess was used. This result suggests that in the gingival pocket fluid of people with periodontitis, about 15% of the proteolytic activity is associated with the participation of enzymes other than cysteine peptidases. The results of these tests are presented in Table 6.

It has been shown that currently highly stable cysteine peptidase inhibitors (several-amino acid oligopeptides and knotweed cystatin), which bind selectively to immobilized papain, inhibit the activity of cysteine gingipain.

From the literature, we know that the activity of the gum-destroying factor, which is induced by two peptidases, gingipain K (Kgp) and R (RgpB and RgpB) decide about 85% of the share in the total proteolytic activity in periodontal flu affected by inflammation. It is suggested that this pathogenic proteolytic activity (85%) is a promising target in the design of new inhibitors precisely inhibiting them (de Diego I, Veillard F, Sztukowska M N, Guevara T, Potempa B, Pomowski A, Huntington J A, Potempa J, Gomis-Rüth F X. (2014): Structure and mechanism of cysteine peptidase gingipain K (Kgp), a major virulence factor of Porphyromonas gingivalis in periodontitis. J Biol Chem. 289, 32291-3302).

It is also known that cystatin from egg white with a concentration of 50 WI is bactericidal against bacteria and can lead to the elimination of up to 50% of them from an infected area of development, e.g. from the periodontium (Blankenvoorde M F, Henskens Y M, van't Hof W, Veerman E C, Nieuw Amteinerongen A V. (1996): Inhibition of the growth and cysteine proteinase activity of Porphyromonas gingivalis by human salivary cystatin S and chicken cystatin. Biol Chem. 377, 847-850; and Blankenvoorde M F, van't Hof W, Walgreen-Weterings E, van Steenbergen T J, Brand H S, Veerman E C, Nieuw Amerongen A V. (1998): Cystatin and cystatin-derived peptides have antibacterial activity against the pathogen Porphyromonas gingivalis. Biol Chem. 379, 1371-1375.)

It is very important that the several-amino acid oligopeptides we isolate from egg and knotweed and cystatin from knotweed are highly stable.

The obtained results may prove that using cystatin from knotweed, egg proteins and several-amino acid inhibitors extracted from the same raw materials, the activity of cysteine gingipain—considered as virulence factor of P. gingivitalis—is blocked. It was also found that the toxicity of cystatin from egg white in chicken embryo studies is about 105 times lower than the toxicity of the approved treatment with 0.02% chlorhexidine.

It can be assumed that both new low-molecular several-amino acid inhibitors and cystatin from knotweed can be used as active agents in pharmaceutical preparations for the treatment and prevention of periodontitis, periodontitis.

Cancer

Autogenic cysteine cathepsins are credited with a significant contribution to the formation and development of cancers in a diseased body. These enzymes are inhibited in vivo by autogenous inhibitors, cystatin. The activity of autogenic cysteine cathepsins is closely related to such a feature of cancer as its aggression, while the level and activity of cystatin depends on the defensive abilities of the sick organism

During cancer, a balance is observed between cysteine activity of cathepsin/cystatin. In a healthy body a balance is observed between the activity of these enzymes and their inhibitors. However, in the case of cancer, the aggression of autogenous cysteine cathepsins begins to achieve an advantage over their inhibitors, which strongly accelerates the development of cancer. Aggression of cancer processes is positively associated with the activity of cysteine cathepsins, while the body's defensive ability is associated with the active level of inhibitors of those enzymes in which the key role is attributed to cystatin C (Kos J, Mitrović A, Mirković B. The current stage of cathepsin B inhibitors as potential anticancer agents. Future Med Chem. 2014 July; 6 (11): 1355-71).

Tumor development, i.e. overexpression of autogenous cysteine cathepsins and exhaustion of the defensive capacity of the sick body suggests that blocking these enzymes should preferably be blocked by using non-toxic specific inhibitors. Numerous attempts are known to use in the inhibition of overexpression of autogenic cysteine cathepsins their synthetic inhibitors, which are unfortunately most often toxic and therefore eliminated from further studies (Löser R, Pietzsch J. 2015 Cysteine cathepsins: their role in tumor progression and recent trends in the development of imaging probes. Front Chem. 23.3, 37, 1-35).

Therefore, the idea arises that cystatin present in natural products should be used to inhibit overexpression of cysteine cathepsins. This approach to controlling the activity of pathogenic cysteine peptidases may prove to be a new, attractive method of therapy.

Information on an efficient method of isolating cysteine peptidase inhibitors is disclosed in EP 2 511 288. Based on this solution, cystatin can be obtained from any natural raw materials. Cystatin from egg white or human placenta was used to inhibit cysteine cathepsin activity. These inhibitors may prove to be factors capable of controlling cancer progression (Saleh Y., Siewiński M., Kielan W., Ziółkowski P. Gryboś M., Rybka J. (2003): Regulation of cathepsin B, L expression in vitro in gastric cancer tissues by egg white cystatin. J. Exp. Ther. Oncol. 319-324; and Basu A, Mills D M, Mitchell D, Ndungo E, Williams J D, Herbert A S, Dye J M, Moir D T, Chandran K, Patterson J L, Rong L, Bowlin T L. (2015): Novel Small Molecule Entry Inhibitors of Ebola Virus. J Infect Dis. 212, S425-434).

When using cystatin from egg white, it was necessary to stabilize it with glycerol or glycols.

Our present invention provides a method for the isolation of new, previously unknown low molecular weight cysteine peptidase inhibitors. They are highly stable several-amino acid peptides, which can be obtained by isolating cystatin from natural raw materials on columns filled with Sepharose-4B-papain. Cystatin and several-amino acid inhibitors of knotweed, sugar cane or sugar beet and mistletoe have also proved to be stable.

Using cystatin from knotweed and several-amino acid inhibitors of knotweed peptidases and egg protein, inhibition of cysteine peptidase activity to about 70% was achieved in lung or colorectal cancer homogenates. The results were obtained and are presented in the above Tables.

Based on the confirmed properties of new inhibitors, it can be anticipated that these inhibitors may be components of future anti-cancer drugs.

Tests were also conducted to determine whether the nebulization process using an ultrasonic inhaler affects the activity of cysteine peptidase inhibitors, which we isolate from natural resources by the method of the invention.

To this end, the activity of cysteine peptidase inhibitors isolated from the knotweed Fallopia japonica, Houtt before and after nebulization were compared, which are:
total inhibitors after separation on an affinity chromatography column,
knotweed cystatin and
several-amino acid peptide knotweed inhibitors.

After isolation of the inhibitors, their saline solutions were nebulized using an ultrasonic inhaler from Beuer I H 40, and then the aerosol mist was condensed using cooled tubes.

The effect of nebulization on the change in the activity of cysteine inhibitors of knotweed from knotweed was determined by comparing the activities of samples of the same inhibitors before and after nebulization, after liquefaction of the aerosols obtained. Solutions after liquefaction were treated as an aerosol, i.e. it was assumed that the composition of the solutions was analogous to the aerosol.

The purpose of this experiment was to show to what extent nebulization changes the activity of cysteine inhibitors of knotweed peptidases in relation to:
papain,
cathepsin B,
trypsin,
gingipains in gingival pockets,
cysteine cathepsins in lung cancer homogenates.

In addition, control of the enzymatic specificity of knotweed inhibitors was introduced by inhibiting them with serine peptidase, native trypsin. The results are shown in Table 7.

Irrespective of the testing of aqueous aerosols, the possibility of using aerosols consisting of glycerin and propylene glycol (as in e-cigarettes) was checked. The e-cigarette stock solution was combined with inhibitors isolated from knotweed and heated to 100° C. Determination of their activity was carried out in samples before and after heating.

To this end, 0.2 ml samples taken from the obtained samples of cysteine inhibitors of peptidases were diluted to 2.0 ml with water (control) and to 2.0 ml with a commercially available solution base for inhalation in e-cigarettes (propylene glycol and vegetable glycerol). Both samples were heated to 100° C. for 10 minutes, then cooled and used to inhibit papain activity on BApNA substrate. The results obtained are shown in Table 8.

Inhibitor Activity Assays

Determination of inhibitors activity against papain: To 1.0 ml of 20 mM pH 6.0 phosphate buffer containing 2.0 mM EDTA and 1.0 mM DTT are added 10 μl papain solution (5 mg/ml), then 20-100 μl inhibitor solution is added followed by 20 μl BApNA substrate solution (Benzoyl-DL Arginyl-para-nitroanilide) 40/mg/ml DMSO. The solution is incubated for 60 min at 37° C., after which the enzymatic reaction is stopped by adding 100 μl of 0.1 M iodoacetic acid. Absorbance is measured at 405 nm on a spectrophotometer relative to the control (without inhibitors and papain). The amount of inhibited enzymatic activity is defined as the inhibitory activity of the tested inhibitors. It is assumed that they are able to inhibit 1 unit of activity of inhibited enzymes (amount of μmol of substrate distributed over 1 minute).

Determination of inhibitors activity against cathepsin B: To 1 ml of 50 mM pH 6.0 phosphate buffer containing 2 mM EDTA and 2 mM DTT are added 10 μl cathepsin B solution (1 mg/ml), then 20-100 μl inhibitor solution and 10 μl Z-Arg substrate solution are added. −Arg-pNA 5/mg/ml DMSO. The solution is incubated for 60 min at 37° C., after which the enzymatic reaction is stopped by adding 100 μl of 0.1 M iodoacetic acid. Absorbance is measured at 405 nm on a spectrophotometer relative to the control (without inhibitors and cathepsin B). The amount of inhibited enzymatic activity is determined by the amount of para-nitroaniline released. As one inhibitory unit is taken the amount of inhibitors that is able to inhibit 1 unit of enzyme activity.

Determination of inhibitors activity against trypsin: To 1.0 ml of 50 mM Tris buffer pH 8.5 is added 10 ml of trypsin solution (5 mg/ml), then 20-100 ml of inhibitor solution, and 10 ml of BApNA substrate solution (Benzoyl-DL Arginyl-para-nitroanilide) 40/mg/ml DMSO. The solution is incubated for 10 min at 37° C., after which the enzymatic reaction is stopped by adding 100 μl of 0.1 M iodoacetic acid. Absorbance is measured at 405 nm on a spectrophotometer relative to the control (without inhibitors and papain). The amount of inhibited enzymatic activity is defined as the inhibitory activity of the tested inhibitors. The amount is taken to be able to inhibit 1.0 unit of activity of inhibited enzymes (the amount of μmol of substrate distributed in 1 minute).

Determination of gingipain activity in fluids taken from gingival pockets: Fluid from the gingival pockets is taken with small strips of cellulose paper, which are placed in a tube containing 1 ml of PBS. The solution is sonicated for 30 sec on ice, then centrifuged at 10,000 g for 10 min.

To 1 ml of 20 mM pH 6.0 phosphate buffer containing 2 mM EDTA and 1 mM DTT is added 100 μl gingipain solution, then 20-100 μL inhibitor solution is added, and 20 μL BApNA substrate solution (Benzoyl DL Arginyl para-nitroanilide) 40/mg/ml DMSO. The solution is incubated for 60 min at 37° C., after which the enzymatic reaction is stopped by adding 100 μl of 0.1 M iodoacetic acid. Absorbance is measured at 405 nm on a spectrophotometer relative to the control (without inhibitors and papain). Gingipain activity is measured by the amount of para-nitroaniline released.

Determination of cathepsin B activity in tissue homogenates: 1.0 g of tissue is placed in a 1 ml PBS tube and sonicated for 30 sec on ice. The suspension is centrifuged at 10,000 g for 19 min. The clear supernatant is used for the assay.

To 1.0 ml of 50 mM pH 6.0 phosphate buffer containing 2 mM EDTA and 2 mM DTT are added 100 μl of homogenate, then 20-100 μl of inhibitor solution are added, and 10 μl of Z-Arg-Arg-pNA substrate solution/mg/ml DMSO. The solution is incubated for 60 min at 37° C., after which the enzymatic reaction is stopped by adding 100 μl of 0.1 M iodoacetic acid. Absorbance is measured at 405 nm on a spectrophotometer relative to the control (without inhibitors and cathepsin B). The amount of inhibited enzymatic activity is determined by the amount of para-nitroaniline released. As one inhibitory unit is taken the amount of inhibitors that it is able to inhibit 1 unit of enzyme activity.

It has now been confirmed that inhibition of undesired overexpression of pathogenic enzymes using aerosols containing inhibitors of the invention is achievable and the results obtained are summarized in Table 7 and Table 8.

TABLE 7
The effect of nebulization on cysteine
peptidase inhibitors from knotweed

| | | | Before nebulization | | After nebulization | |
|---|---|---|---|---|---|---|
| | Material | | Activity | % inhi- | Activity | % inhi- |
| No. | tested | Inhibitors | U/ml | bition | U/ml | bition |
| 1. | Papain | 0 | 1.87 | 0.00 | 1.87 | 0.00 |
| | | The lot | 0.37 | 80 | 0.41 | 78 |
| | | Cystatin | 0.45 | 76 | 0.48 | 74 |
| | | Peptides | 0.52 | 72 | 0.60 | 68 |
| 2. | Trypsin | 0 | 2.43 | 0.00 | 2.43 | 0.00 |
| | | The lot | 2.35 | 3 | 2.36 | 3 |
| | | Cystatin | 2.38 | 2 | 2.39 | 2 |
| | | Peptides | 2.41 | 2 | 2.40 | 2 |
| 3. | Cathepsin B | 0 | 1.32 | 0 | 1.32 | 0 |
| | | The lot | 0.44 | 67 | 0.46 | 65 |
| | | Cystatin | 0.48 | 64 | 0.50 | 62 |
| | | Peptides | 0.52 | 61 | 0.55 | 58 |
| 4. | Lung | 0.00 | 0.82 | 0 | 0.82 | 0 |
| | cancer 1 | The lot | 0.24 | 71 | 0.26 | 68 |
| | | Cystatin | 0.28 | 66 | 0.31 | 62 |
| | | Peptides | 0.32 | 61 | 0.35 | 57 |
| 5. | Lung | 0 | 0.65 | 0 | 0.65 | 0 |
| | cancer 2 | The lot | 0.14 | 79 | 0.16 | 75 |
| | | Cystatin | 0.16 | 75 | 0.18 | 72 |
| | | Peptides | 0.19 | 71 | 0.24 | 63 |
| 6. | Gingipains 1 | 0 | 0.92 | 0 | 0.92 | 0 |
| | | The lot | 0.16 | 83 | 0.21 | 77 |
| | | Cystatin | 0.25 | 73 | 0.28 | 70 |
| | | Peptides | 0.22 | 76 | 0.25 | 73 |
| 7. | Gingipains 2 | 0 | 0.98 | 0 | 0.98 | 0 |
| | | The lot | 0.21 | 82 | 0.24 | 75 |
| | | Cystatin | 0.27 | 74 | 0.29 | 71 |
| | | Peptides | 0.29 | 70 | 0.33 | 68 |

TABLE 8
The effect of 100° C. temperature on the activity
of knotweed cysteine inhibitors in water and propylene-
glycerol glycol on inhibition of papain activity

| | | | | | After heating | |
|---|---|---|---|---|---|---|
| | | | Before heating | | at 100° C. | |
| | Material | | Activity | % inhi- | Activity | % inhi- |
| No. | tested | Inhibitors | U/ml | bition | U/ml | bition |
| 1. | Inhibitors in | | 1.68 | 0 | 1.68 | 0 |
| | 90% base | Eluate | 0.31 | 82 | 0.41 | 75 |
| | solution | from the | | | | |
| | (glycol- | column | | | | |
| | glycerol) | Cystatin | 0.41 | 75 | 0.49 | 71 |
| | | Peptides | 0.48 | 71 | 0.62 | 63 |
| 2. | Inhibitors in | | 1.68 | 0 | 1.68 | 0 |
| | aqueous | Eluate | 0.43 | 74 | 0.43 | 74 |
| | solution | from the | | | | |
| | | column | | | | |
| | | Cystatin | 0.51 | 70 | 0.51 | 70 |
| | | Peptides | 0.62 | 62 | 0.62 | 62 |

In accordance with the invention, inhibitory possibilities have been provided by natural inhibitors of overexpression of cysteine enzymes of peptidases derived from both pathogenic microorganisms and autogenous (human and animal) genetically altered cells, including cancer cells.

As a preferred route of administration we present the method of transferring peptide cysteine inhibitors of peptidases isolated on the basis of our method from natural raw materials to places where inflammation develops. The main target organs are the oral cavity and the respiratory system, in which overexpression of cysteine peptidases associated with these conditions occurs.

Inhibitors administered in this way will block pathogenic enzymes derived both from the human/animal's own cells, as cysteine cathepsins or from microorganisms, exemplified by gingipaines from the bacterium Porphyromonas gingivitalis occurring in periodontitis.

Using the aerosol to transport inhibitors according to the invention, we plan to deliver inhibitors to the entire oral cavity, sinuses and the upper and lower respiratory system. Overexpression of cysteine peptidases in inflammation can be caused by both enzymes from pathogenic microorganisms, such as viruses (influenza), bacteria (periodontitis) and other or cathepsins originating from own cells (malignant tumors).

The methods of producing aerosols, i.e. so-called nebulization, have been well known and can be used in the targeted transport of inhibitors from the technical point of view. They ensure non-invasive delivery of medicinal substances to an important area in the human/animal body extremely susceptible to inflammation, which is the area of the mouth and respiratory system.

It is a way of administering non-toxic cysteine inhibitors of peptidases to sites affected by inflammatory changes in the human body, which the aerosol can reach without hindrance and inhibit the activity of pathogenic enzymes

In the course of the work on the present invention, it has been shown that nebulization of cysteine inhibitors of knotweed peptidases (Fallopia japonica, Houtt) including cystatin, low-molecular peptides and their mixture obtained directly after affinity chromatography does not significantly change the inhibitory activity compared to the inhibitory activity before nebulization.

The activity of inhibitors after nebulization and then after its liquefaction was measured relative to native cathepsin B and papain and gingipain in gingival pockets of patients with periodontitis (periodontitis) and cysteine cathepsins in lung cancer tissue homogenates. The activity of the tested inhibitors after nebulization showed a slightly lower level compared to the activity before nebulization. The results are shown in Table 7 above.

It was assumed that the activities of inhibitors obtained as a result of liquefaction of aerosols are the same as when they were sprayed to specific locations throughout the entire oral cavity and respiratory tract.

The inhibitors contained in the aerosol are designed to reach areas with inflammation that are overexpressing cysteine peptidases and lead to inhibition of these enzymes. The inhibitory effect can be treated as a therapeutic effect.

Administration of the drug substance by nebulization is an uncomplicated and simple method.

In the in vitro studies, we chose gingipain as the frontal virulence factors of the pathogenic Porphyromonas gingivitalis oral bacterium and cysteine cathepsins present in lung cancer homogenates as potential targets for inhibitors (after nebulization and subsequent liquefaction). Cathepsin B in lung cancer, like gingipain in periodontitis, are considered as potential therapeutic targets for these diseases. (Olsen I, Potempa J. 2014 Strategies for the inhibition of gingipains for the potential treatment of periodontitis and associated systemic diseases. J Oral Microbiol. 6: doi: 10.3402/jom.v6.24800.; Gondi C S, Rao J S. 2013 Cathepsin B as a cancer target. Expert opinions and goals. 17; 281-291. Doi: 10.1517/14728222.2013.740461).

In the course of research on the invention, inhibitors that were initially nebulized and then liquefied also inhibited the activity of enzymes such as cathepsin B and papain and trypsin. The latter enzyme was selected to confirm that the currently isolated inhibitors of the invention react specifically with cysteine peptidases.

Based on the results obtained, it is possible to state the ability to inhibit cysteine peptidases by inhibitors of the invention subjected to nebulization, and the lack of inhibition of serine peptidases, indicates the selective action of the cysteine inhibitors of the peptidases of the invention.

The choice of the area of the respiratory system to which the indicated inhibitors are delivered can be controlled by the size of the aerosol droplets. Their size is influenced by the arrangement and selection of inhalers, which allows obtaining the right size droplets. This allows you to prepare inhalation for selected areas of the human respiratory system.

Aerosols can be used as a complement to methods of combating the dangerous pathogen of P. gingivitalis in the gum area. The gingipain exoenzymes of this bacterium lead to periodontal degradation called periodontitis. In addition to changes in the periodontium itself, this disease leads to very serious systemic diseases. For this reason, the bacterium that causes it is referred to as one of the most dangerous oral pathogens. Its elimination can protect both the periodontium and the body against the appearance of systemic diseases caused by it, such as: arteriosclerosis, pregnancy complications, diabetes, gastrointestinal cancer, Alzheimer and others. (Carter C J, France J, Crean S, Singhrao S K. 2017 Porphyromonas gingivalis/Host Interactome Shows the enrichment in GWASdb of genes associated with Alzheimer's disease, diabetes and cardiovascular disease. Aging neurosis in front. 9: 408. 12.12 doi: 10.3389/fnagi.2017.00408; Olsen I, Yilmaz Ö. 2019 Possible role of Porphyromonas gingivalis in oral cancer. J Oral Microbiol. 11: 1563410. doi: 10.1080/20002297.2018.1563410).

Research on the present invention shows that cysteine peptidase inhibitors isolated from knotweed effectively inhibit proteolytic activity in gingival pocket fluids by about 85%, suggesting inhibition of gingipain activity, which is decisive for degradation of periodontal tissues. A similar result was obtained by inhibiting the same activity with cystatin from egg white.

It turned out that both knotweed inhibitors and cystatin from egg white inhibit the proteolytic activity of gingival pocket fluids to the same extent. At the same time, it has been shown that the same proportion of proteolytic activity in gingival pocket fluids is attributed to P. gingivitalis gingipaines. Cysteine peptidases have been shown to be key factors in the proteolytic pathogenicity of this bacterium that causes chronic periodontitis, the most common disease caused by dysbiosis in humans. The two peptidases, gingipaina K (Kgp) and R (RgpA and RgpB), which differ in selectivity after lysine and arginine, respectively, account for 85% of the extracellular proteolytic activity of P. gingivalis at the site of infection (de Diego I, Veillard F, Sztukowska M N, respectively), et al. 2014 Structure and mechanism of cysteine peptidase gingipain K (Kgp), a major virulence factor of Porphyromonas gingivalis in periodontitis. J Biol Chem. 289; 32291-32302. doi: 10.1074/jbc.M114.602052). This is the same share in total proteolytic activity in periodontal pockets of patients with periodontitis.

Based on this information, it can be suggested that the same percentage of proteolytic activity that is attributed to enzymes produced in one of the most virulent oral pathogens, P. gingivitalis, i.e. 85%, is inhibited with the help of the cysteine inhibitors of the peptidases of the invention obtained from knotweed and egg protein.

The isolated methods of the invention inhibitors of cysteine peptidases (cystatin and several-amino acid peptides) that inhibit pathogenic cysteine peptidases in the oral cavity such as gingipain (paradontosis) and cysteine cathepsin (lung cancer tissue) will be the basis of aerosol inhibition.

Bacterial enzymes (periodontitis) and autogenous as cysteine cathepsins (lung cancer) were selected as examples of inhibited enzymes for current research. Table 7 shows the results of inhibition of these enzymes by cysteine peptidase peptidase inhibitors previously nebulized in an ultrasonic inhaler. It turns out that switching them to an aerosol does not affect any changes in the activity of these inhibitors. Both before and after entering the aerosol form, their activity in relation to gingipain (periodontitis), cysteine cathepsins (lung cancer tissues), as well as native cathepsin B, papain or trypsin are inhibited practically the same before and after nebulization—Table 7. Confirms that cysteine peptidase inhibitors from natural raw materials in aerosols will show the ability to inhibit overexpression of pathogenic cysteine peptidases throughout the entire mouth and respiratory tract.

The use of cysteine inhibitors of knotweed or other natural raw materials administered in the form of aerosols can help in the elimination of P. gingivitalis from such an area of the entire oral cavity that is practically inaccessible to toothpaste, gels for rubbing gums or rinses. Aerosols with inhibitors may therefore find application as complementary or alternative methods to conventional products used to maintain oral hygiene and locally protect it against periodontitis and caries, as well as methods based on the use of chlorhexidine or tetracycline for local destruction of bacterial flora, including P. gingivitalis (Shantipriya R., Prasad M G S., Bhowmick N., Singh S., Amir A., Vimal S K. 2016 A comparison of Chlorhexidine and Tetracycline local drug delivery systems in management of persistent periodontal pockets—A clinical study. Int. J. Appl. Dent. Sci. 2; 11-15; Kumar A J, Ramesh Reddy B V, Chava V K. 2014 The effect of chlorhexidine chip in the treatment of chronic periodontitis. J Nat Sci Biol Med. 5; 268-272. Doi: 10.4103/0976-9668.136159).

P. gingivitalis bacteria that inhabit the tongue, cheeks and other oral tissues pose a very serious problem in achieving full cure of periodontitis. No comprehensive way of destroying them has yet been discovered. Until now, tetracyclines and chlorhexidine could not be used in a wider range to fully destroy this bacterium, because the use of the above-mentioned aerosol, which has access to the entire area of the mouth and respiratory system, could have caused a strong toxicity effect (chlorhexidine) or drug resistance of the bacterial flora in the cavity oral (tetracycline).

The delivery of natural cysteine peptidase inhibitors in the form of inhalation in accordance with the present invention provides the possibility of comprehensive disposal of P. gingivitalis.

It is known that these inhibitors can be nebulized and sprayed because they retain their original properties without causing a toxic effect. Without going into the actual mechanisms of action, it should be presumed that these inhibitors do not act directly on pathogenic microbial cells, including P. gingivitalis that produces gingipaines, but block access to the substances/energy they need to survive. Similar effects also appear after contact with other pathogens. Providing natural cysteine peptidase inhibitors in inhalations from the oral cavity and the respiratory tract creates a chance for practical use of aerosols for the selective elimination of pathogenic microorganisms from this area of the human body, which is highly exposed to infections.

To date, participation in the destruction of the pathogenic P. gingivitalis oral bacterium using natural egg white cystatin has been investigated. (Blankenvoorde M F, van't Hof W, Walgreen-Weterings E. et al. 1998 Cystatin and cystatin-derived peptides have antibacterial activity against the pathogen Porphyromonas gingivalis. Biol Chem. 379, 1371-1375).

We do not exclude the use of natural cysteine inhibitors of peptidases isolated from other natural raw materials, provided they are not toxic and stable.

The research also found that inhibitors isolated according to the invention retain their activity in solution, which can form the basis for e-cigarettes both at room temperature and when heated to 100° C. It can therefore be assumed that there is a possibility of transferring them in an aerosol that will include glycerol and propylene glycol used in e-cigarettes (NIDA. “Tobacco/nicotine and vaping.” National Institute on Drug Abuse, https://www.drugabuse. gov/drugs-abuse/tobacconicotine-vaping; Kuntawala S., Moy L., Patel A. 2016 E-cigarette or vaping fluid. U.S. Pat. No. 0,198,759 A1).

The destructive processes in the gums are associated with the activation of pathogenic peptidases, including cysteine gingipain secreted by bacteria responsible for the development of chronic inflammation.

It turned out that in patients with periodontitis, the activity of cystatin decreases significantly compared to the corresponding group of people with a healthy periodontium. Excess gingipain causes these inhibitors to bind, hence their significant decrease in activity.

Saliva produced by salivary glands contains biologically active proteins responsible for controlling the changes in the mouth of humans and animals. Of the cystatins present in saliva, gingipain activity in the gingival pocket fluid is inhibited only by cystatin S and cystatin from egg white. These are natural cysteine peptidase inhibitors that were previously (PL 219098) used to remove the effects of cysteine peptidase activity in periodontal disease.

Cystatin from egg white under native conditions has low stability due to the fusion of molecules into dimers. Therefore, cystatin in 20% glycerol should be suspended for application. In this form, cystatin from eggs retains activity and is suitable for use in the preparation of the medicament according to the invention, useful in the prevention and treatment of periodontitis in humans and companion animals, especially dogs.

It has now surprisingly been found that very good therapeutic effects are obtained by using a lower concentration of cystatin in the composition of the medicament according to the invention, useful in the prevention and treatment of periodontitis in humans and companion animals, especially in dogs. In addition, traditional topical use of cystatin is supplemented by oral administration in the form of aqueous solutions.

According to the invention, a much simpler composition containing low concentrations of cystatin (10-200 mU in 100 ml) as an inhibitor of cysteine gingipain secreted by bacteria that cause periodontal disease, in a water-glycerin solution stabilized with sodium benzoate effectively inhibits the activity of cysteine gingipain in the gingival pockets of patients—both people and dogs.

Further details of the invention are disclosed in the following examples.

### Example 1. In Vivo Inhibition of Cysteine Gingipain Activity in Dogs

A solution containing known cystatin from egg white with a concentration of 0.02% and activity of 12 U/mg and 20% glycerin was used to rub the gums of dogs (once a day) and added 0.2 ml to a bowl with 200 ml drinking water (1 mU)/ml cystatin) for 4-6 weeks. Liquids from gingival pockets were removed using plaque paper, which was placed in 1 ml saline and frozen at −80° C., before removing plaque calculus. Then, after the aforementioned cystatin therapy from egg white, fluids from the gingival pockets were taken again in an analogous manner and frozen. After obtaining the desired amount of samples, they were thawed, sonicated, centrifuged to then determine the activity of cysteine gingipain in 100 μL samples using a BANA substrate (Benzoyl-arginyl-beta-naphthylamide). Test samples with a volume of 100 μl were preincubated at 37° C. in 0.01 M phosphate buffer pH 6.0 with the addition of 1 μM DL-cysteine and 2 μM EDTA for 10 minutes, then a solution of N-benzoyl DL-arginyl hydrochloride-β-naphthylamide (BANA) so that its final concentration in the test sample was 1.5 mM, and then incubated under these conditions for 30 minutes. After this time, the reaction was stopped by determining the colorimetrically released β-naphthylamine.

Standard tests for which the final result was determined were prepared in parallel with the tested samples, which were solutions containing all components as in the actual tests, except that after adding the BANA substrate the reactions were immediately stopped and the colorimetrically determined free β-naphthylamine was determined. As one unit of enzymatic activity of cysteine proteinases is the amount of enzyme which releases 1.0 nmole of β-naphthylamine from the substrate within one hour under the described incubation conditions, whereas as an inhibitor unit—the amount that inhibits 1 enzyme unit.

Approximately 67% of cysteine gingipain activity was inhibited in the gingival pocket fluids of the test animals. The results obtained are summarized in Table 9).

TABLE 9
Effect of inhibiting the activity of pathogenic
cysteine gingipain in fluid samples from
gingival pockets having rubbed gums with a cystatin
solution from egg white

| | Activity | Activity | | |
|---|---|---|---|---|
| | before | after | % | % |
| Sample | plaque | cystatin | remaining | inhibited |
| number | removal | treatment | activity | activity |
| 1 | 1.09 | 0.06 | 5.51 | 94.79 |
| 2 | 0.69 | 0.16 | 22.69 | 77.31 |
| 3 | 0.49 | 0.10 | 20.77 | 79.23 |
| 4 | 0.46 | 0.19 | 41.10 | 58.9 |
| 5 | 0.17 | 0.061 | 35.67 | 64.33 |
| 6 | 1.05 | 0.07 | 6.50 | 93.50 |
| 7 | 0.07 | 0.05 | 75.76 | 24.24 |
| 8 | 0.73 | 0.14 | 18.73 | 81.26 |
| 9 | 0.62 | 0.20 | 32.32 | 67.68 |
| 10 | 0.12 | 0.05 | 45.22 | 54.78 |
| 11 | 0.80 | 0.47 | 58.45 | 41.55 |
| | | Average | 32.97 ± 21.49 | 67.05 ± 21.52 |

### Example 2. In Vitro Inhibition of Cysteine Proteases in Dogs

Before periodontal removal, dogs with periodontal disease collected fluid from the gingival pockets using tissue strips that were placed in 1 mL of saline. The solution was sonicated, centrifuged and 100 μl of supernatant was taken for assay. Various amounts of cystatin were added to the test solution, and then the protease activity was determined using the BANA substrate as in Example 1. About 40 ng of cystatin was found to be able to inhibit over 90% of activity in all gingival fluid. In the group of 19 dogs, the average inhibition of activity was 87%. The results of the conducted tests are summarized in Table 10.

TABLE 10
Ability to maximal inhibition of cysteine gingipain activity in
dogs gingival pockets after administration of 40 ng cystatin
from egg white with an activity of 12 units/mg protein

| | Cysteine | Cysteine | | |
|---|---|---|---|---|
| | gingipains | gingipain | | |
| | activity before | activity after | % | % |
| Sample | plaque | adding | remaining | inhibited |
| number | removal | cystatin | activity | activity |
| 1 | 1.09 | 0.06 | 5.30 | 94.70 |
| 2 | 0.69 | 0.04 | 6.21 | 93.79 |
| 3 | 0.49 | 0.03 | 6.52 | 93.48 |
| 4 | 0.46 | 0.08 | 17.80 | 82.20 |
| 5 | 0.17 | 0.04 | 23.98 | 76.02 |
| 6 | 1.05 | 0.03 | 2.49 | 97.51 |
| 7 | 0.07 | 0.02 | 27.27 | 72.73 |
| 8 | 0.73 | 0.04 | 4.82 | 95.18 |
| 9 | 0.62 | 0.07 | 8.84 | 91.16 |
| 10 | 0.12 | 0.03 | 26.96 | 73.04 |
| 11 | 0.79 | 0.02 | 2.38 | 97.62 |
| 12 | 1.29 | 0.09 | 6.61 | 93.39 |
| 13 | 1.16 | 0.15 | 13.14 | 86.86 |
| 14 | 0.87 | 0.06 | 7.33 | 92.67 |
| 15 | 0.92 | 0.24 | 26.17 | 73.83 |
| 16 | 0.87 | 0.07 | 7.69 | 92.31 |
| 17 | 1.03 | 0.13 | 13.07 | 86.93 |
| 18 | 0.92 | 0.23 | 25.35 | 74.65 |
| 19 | 1.36 | 0.13 | 9.69 | 90.32 |
| | | Average | 12.72 ± 8.92 | 87.28 ± 8.92 |

### Example 3. In Vitro Inhibition of Cysteine Gingipain Activity in Humans

Patients with periodontitis were taken from the gingival pockets before removing the plaque using tissue strips, which were placed in 1 ml of saline and then frozen at −80° C. After collecting the appropriate number of samples, the solutions were sonicated, centrifuged and 100 μl of cysteine gingipain activity was taken for determinations using BANA as a substrate as in Example 1. The solution was sonicated, centrifuged and 100 μl of supernatant was taken for determinations. Various amounts of cystatin were added to the test solution, and then the protease activity was determined by the BANA test as in Example 1. It was shown that about 200 ng of cystatin is able to inhibit about 90% of the activity in the entire gingival fluid. In the group of 23 patients, the average inhibition of activity was 83%. The obtained results are collected in Table 11).

TABLE 11
Activity of cysteine gingipain in examined gingival
pocket fluids in patients with periodontitis

| | | CG activity | | |
|---|---|---|---|---|
| | Activity of | after the | % | % |
| | cysteine | addition of | remaining | inhibited |
| Sample | gingipains | cystatin | activity | activity |
| number | [U/ml] | [U/ml] | [%] | [%] |
| 1 | 2.57 | 0.47 | 18.29 | 81.71 |
| 2 | 2.17 | 0.61 | 28.11 | 71.89 |
| 3 | 10.95 | 1.16 | 10.59 | 89.41 |
| 4 | 9.72 | 0.98 | 10.08 | 89.92 |
| 5 | 4.17 | 1.12 | 26.86 | 73.14 |
| 6 | 3.96 | 0.75 | 18.94 | 81.06 |
| 7 | 3.53 | 0.34 | 9.63 | 90.37 |
| 8 | 8.29 | 0.47 | 5.67 | 94.33 |
| 9 | 7.89 | 0.87 | 11.03 | 88.97 |
| 10 | 8.25 | 1.22 | 14.79 | 85.21 |
| 11 | 9.18 | 0.98 | 10.68 | 89.32 |
| 12 | 7.76 | 1.11 | 14.30 | 85.70 |
| 13 | 5.23 | 0.47 | 8.99 | 91.01 |
| 14 | 14.84 | 2.19 | 14.76 | 85.24 |
| 15 | 9.77 | 1.12 | 11.46 | 88.54 |
| 16 | 11.68 | 2.52 | 21.57 | 78.43 |
| 17 | 7.93 | 1.97 | 24.84 | 75.16 |
| 18 | 16.51 | 3.96 | 24.00 | 76.00 |
| 19 | 9.82 | 2.18 | 22,.0 | 73.80 |
| 20 | 12.71 | 3.11 | 24.47 | 75.53 |
| 21 | 9.23 | 1.28 | 13.87 | 83.13 |
| 22 | 13.63 | 2.74 | 20.10 | 79.90 |
| 23 | 10.28 | 1.73 | 16.83 | 83.17 |
| | | Average: | 15.89 ± 6.28 | 83.08 ± 6.63 |

### Example 4. Dog Case Study

A female mixed breed, weighing about 5 kg and 7 years old, became dejected and avoided food, staggered and turned over, and purulent discharge leaked from her nose. Only a visit to another clinic showed the correct cause of this condition, which was caused by high levels of tartar. In dormancy, the stone was removed along with four molars on the left. During sleep, gingival pocket fluids were removed from 3 gingival pockets on the right using strips of paper and placed in tubes containing 1 ml of saline. After grinding the paper with ultrasound and centrifugation, the activity of cysteine gingipain was determined in solutions. Their activity ranged from 1.35 to 0.89 units based on 1.0 ml of liquid based on the amount of β-naphthylamine released. For 4 weeks, the dog was given water to drink with the addition of cystatin from egg protein at a concentration of about 10-6% and rubbed the gums with a pad impregnated with 0.001% cystatin from egg white 3-4 times a day. After 3 months, test fluids were again taken from the same gingival pockets and gingipain activity was found to be reduced almost five-fold. There was also no tartar build-up or redness in the gums. After 6 years of surgery, there were no changes in the periodontium. After some time, the dog got used to rubbing his gums, which probably saved him from changes in the periodontium.

### Example 5. Isolation of Several-Amino Acid Peptides from Knotweed (Fallopia japonica, Houtt.)—Case Study

The first stage of isolation of cysteine peptidases was performed as described in patent specification EP 2 511 288. Green knotweed (Fallopia japonica, Houtt.) With a mass of 500 g was placed in 1000 ml of distilled water, and then after comminution in a homogenizer, the homogenate was adjusted to pH 2.0 using 0.1 M hydrochloric acid, heated to 90° C. for 20 minutes, cooled to room temperature, neutralized with 0.1 M NaOH and frozen for 24 hours at −20° C. After thawing, the whole was centrifuged at 25,000 rpm for 20 minutes, and 50 ml of 0.1 M phosphate buffer pH=6.8 was added to the obtained supernatant. This solution was additionally filtered on paper filters and applied to a column containing 500 cm3 of Sepharose 4 B carrier—papain, washing the column initially with 0.05 M NaCl and then with distilled water until the protein in the eluent disappears. The washing solution was then changed to 0.01 M NaHCO₃ at pH=11.0. Protein-containing fractions were preferably collected into tubes using a collector. Up to this stage, the procedure was in accordance with the procedure set out in patent no EP 2 511 288. According to the present invention, there is no need to add cystatin stabilizers such as glycerol or polyglycols to the eluent.

The next step was the isolation of several-amino acid peptide inhibitors. When the protein from the column stopped flowing, the fractions containing them were collected into a beaker without adding glycerol or polyglycols to stabilize cystatin and passed through a 3 kDa membrane that passes several-amino acid oligopeptides capable of inhibiting papain and retaining cystatin. The extracted several-amino acid oligopeptides are stable at room conditions and no stabilizers such as glycerol had to be added.

The ability to inhibit selected cysteine peptidases in solutions before and after the membrane was then determined. About 13 Da cystatin remains in front of the membrane, and in the solution after the 3 kDa membrane—several-amino acid oligopeptides.

Knotweed cystatin as well as several-amino acid oligopeptides retained their inhibitory activity even after storage for several months in the refrigerator. Under such conditions, cystatin from egg protein dimerizes and loses its biological activity. Inhibitory activity of cystatin and several-amino acid inhibitors of cysteine peptidases were determined in relation to cysteine gingipain in gingival pocket fluids and cysteine cathepsins in cancerous tissues and veins of people with atherosclerosis or native cathepsin B.

1. Low molecular weight cysteine peptidase inhibitors of natural origin, which are several-amino acid oligopeptides with a molecular weight below 3 kDa.

2. Low molecular weight cysteine peptidase inhibitors according to claim 1, characterized in that they are free of salts having a molecular weight below 700 Da.

3. Low molecular weight cysteine peptidase inhibitors according to claim 1 or 2, characterized in that they are obtained from a group of natural raw materials, which includes egg protein, casein, milk, knotweed, especially of the species Fallopia japonica, Houtt, mistletoe, soybean, pineapple, rice, potatoes and other substances of natural origin.

4. Cystatin derived from knotweed, especially from the species Fallopia japonica, Houtt, stable in a glycerol and other glycols free environment.

5. A method of isolating low-molecular weight cysteine peptidase inhibitors from natural organic materials, in which a protein mass is prepared by grinding and/or mixing with water plant material, casein, milk or egg protein, then the pH of the aqueous mixture is lowered to about 2 and the whole mass is subjected for a short time to a high temperature below the water boiling point, and then after freezing the whole mass for at least few hours and thawing it, by means of affinity chromatography with a carrier with cysteine peptidases bonded thereto, cysteine peptidase inhibitors are trapped on this carrier which are then eluted from the chromatographic column, characterized in that the eluent from the affinity chromatography column is collected by means of a fraction collector, next the fractions showing the presence of protein compounds are collected and combined, and then the combined protein eluates are passed through a membrane cutting off compounds larger than 3 kDa and the fraction passing through the membrane is desalted by filtering it through a membrane passing compounds smaller than 700 Da.

6. The method according to claim 5, characterized in that the protein mass obtained from egg whites diluted with a distilled water, preferably in a weight ratio of egg mass to water being 1:1 or from other biological material selected from the group consisting of casein, milk and plant homogenates such as knotweed, especially of the species Fallopia japonica, Houtt, mistletoe, soybean, pineapple, rice, potatoes and other substances of natural origin.

7. Medical use in human and veterinary medicine of the low molecular weight inhibitors of cysteine peptidases of natural origin, being several-amino acid oligopeptides of the molecular weight below 3 kDa, as defined in claims 1-3 and cystatin as defined in claim 4, as a therapeutically and/or prophylactically active substance.

8. Use according to claim 7, of the low molecular weight cysteine peptidase inhibitors of natural origin, being several-amino acid oligopeptides of the molecular weight below 3 kDa, and of the said cystatin, for manufacturing the medicaments useful in prevention and treatment of periodontitis, in human and veterinary medicine.

9. Use according to claim 7, of the low molecular weight cysteine peptidase inhibitors of natural origin, being several-amino acid oligopeptides of the molecular weight below 3 kDa, and of the said cystatin, for manufacturing the medicaments useful in prevention and treatment of malignant tumors accompanied by cathepsins.

10. Use according to claim 7, of a mixture of cysteine peptidase inhibitors obtained by the method of claim 5, after affinity chromatography, from knotweed or other plants, or raw materials obtained from vertebrates, for manufacturing medicaments useful in prevention and treatment of periodontitis, in human and veterinary medicine.

11. Use according to claim 7, of a mixtures of cysteine peptidase inhibitors obtained by the method of claim 5, after affinity chromatography, from knotweed or other plants, or raw materials obtained from vertebrates, for manufacturing medicaments useful in prevention and treatment of malignant tumors accompanied by cathepsins.

12. A medicament for use in human and veterinary medicine, in the form of an aqueous aerosol or of a propylene glycol and plant glycerol aerosol (e-cigarette aerosol) containing an effective amount of the low molecular weight inhibitors of cysteine peptidases, of natural origin, being several-amino acid oligopeptides of the molecular weight below 3 kDa, as defined in claims 1-3 and the cystatin as defined in claim 4, intended for inhalations and administration to the oral cavity, sinuses and upper and lower respiratory tract, in prevention and treatment of inflammations associated with overexpression of cysteine peptidase enzymes originating from infecting microorganisms, or with overexpression of cysteine peptidase enzymes, being autogenic cysteine cathepsins in the human body.

13. A medicament for use in human and veterinary medicine, in the form of an aqueous aerosol or of a propylene glycol and plant glycerol aerosol (e-cigarette aerosol) containing an effective amount of a mixture of cysteine peptidase inhibitors obtained by the method of claim 5, after affinity chromatography, from knotweed or other plants or raw materials obtained from vertebrates, intended for inhalations and administration to the oral cavity, sinuses and upper and lower respiratory tract, in prevention and treatment of inflammations associated with overexpression of cysteine peptidase enzymes originating from infecting microorganisms, or with overexpression of cysteine peptidase enzymes, being autogenic cysteine cathepsins in the human body.

14. A medicament for use in human and veterinary medicine, in the form of an aqueous aerosol or of a propylene glycol and plant glycerol aerosol (e-cigarette aerosol) containing an effective amount of the several-amino acid peptides having the of cysteine peptidase inhibitors properties the peptides obtained by chemical synthesis to meet the pattern of the oligopeptides of the molecular weight below 3 kDa, as defined in claims 1-3 and the cystatin as defined in claim 4, intended for inhalations and administration to the oral cavity, sinuses and upper and lower respiratory tract, in the treatment and prevention of inflammations associated with overexpression of cysteine peptidase enzymes originating from infecting microorganisms, or with overexpression of cysteine peptidase enzymes, being autogenic cysteine cathepsins in the human body.

15. A medical device in the form of an inhaler device, preferably an ultrasonic inhaler device for administering a therapeutic agent (medicament) according to the claims 12 to 14.
